(19) United States
(12) Patent Application Publication
(10) Pub. No.: US 2021/0315251 A1
(43) Pub. Date: Oct. 14, 2021

(54) FOOD ADDITIVE AND METHOD FOR MODULATING GUT MICROBIOTA PROFILE

(71) Applicant: UNIVERSITY OF MARYLAND, COLLEGE PARK, COLLEGE PARK, MD (US)

(72) Inventors: LIANGLI YU, FULTON, MD (US); ELENA BAILONI, LEWISBERRY, PA (US); UYORY CHOE, HYATTSVILLE, MD (US); YANFANG LI, COLLEGE PARK, MD (US)

(21) Appl. No.: 17/301,648

(22) Filed: Apr. 9, 2021

Related U.S. Application Data
(60) Provisional application No. 63/007,734, filed on Apr. 9, 2020.

(51) Int. Cl. A23L 33/135; A23L 33/105; A23L 29/238

(52) U.S. Cl. CPC A23L 33/135; A23L 29/238; A23L 33/105

(57) ABSTRACT

Tomato seed flour and oil were evaluated for the chemical composition, Total Phenolic Content and GUT microbiota alterations indicative of radical scavenging and anti-inflammatory capacities to validate the potential as a health-beneficial value-added food. It was proven that tomato seed flour altered GUT microbiota profile in vitro. Identifying tomato seed flour as a value-added product can reduce waste and increase the profits for businesses while improving human health. Although tomato seed flour showed greater amount of beneficial compounds than the tomato seed oil, there is still a potential for the use of tomato seed oil in altering the microbiota profile in various ways.

### REFERENCE TO THE RELATED APPLICATION

This Utility Patent Application is based on the Provisional Patent Application No. 63/007,734 filed on 9 Apr. 2020.

### FIELD OF THE INVENTION

The present invention is directed to improving the health of a user consuming a seed powder/meal, and/or seed oil and/or seed flour which are capable of modulating microbiota in a user's gastrointestinal tract (GUT).

The present invention is also directed to seed powder/meal, and/or seed oil and/or seed flour which may be produced by processing by-products of plants, such as fruits, vegetables, and berries, for example, the seeds of tomatoes and/or blackberries.

In overall concept, the present invention is directed to modulating of the GUT microbiota profile to reduce oxidative stress and inflammation, to attain reduction of the risk of obesity, diabetes, and other chronic diseases, by consumption of seed powder/meal and/or seed oil and/or seed flour, which are derivatives of processing of seeds of vegetables, fruits, and berries, and which are validated for effective biological modulating of GUT microbiota profile.

In addition, the present invention is directed to seed powder/meal and/or seed oil and seed flour which may be derived from one or more plant, for example, tomato seeds, and which may be used as nutraceuticals, or bioactive food ingredients, functional foods or dietary supplements having free radicals scavenging and anti-inflammatory capacities and an increased Total Phenolic Content which has beneficial effects in scavenging of free radicals.

Furthermore, the present invention is directed to tomato seed oil which shows promising results in GUT microbiota profile alteration and reducing body weight, where the tomato seed oils and flours would be used for disease prevention and health promotion as pre-biotics.

The present invention also addresses the study of the tomato seed flour and tomato seed oil and validation of these seed derivatives for modulation of GUT microbiota profile as well as free radicals scavenging and anti-inflammatory capacities, as well as control of body weight.

### BACKGROUND OF THE INVENTION

The gastrointestinal tract (GUT) microbiota profile is an important factor for the state of human health. Each bacterial phylum or genus has its own role in the human body system. Therefore, maintaining a healthy GUT microbiota profile through dietary intervention is important.

Phenolic compounds, i.e., one group of the compounds found in healthy foods, such as fruits, vegetables, and berries are known to potentially interact with GUT microbiota. In addition, phenolic compounds are strong free radical scavengers, which may prevent excessive accumulation of free radicals in the human body. Excessive radicals may cause a number of chronic diseases including obesity, celiac disease, cardiovascular disease, type 2 diabetes, and cancers. Thus, scavenging free radicals through dietary intervention is essential for improving human health.

In addition, a healthy diet can suppress or block the transcriptional level of pro-inflammatory genes and significantly reduce the risk of human chronic diseases.

A number of studies on edible seed flours have been conducted, which resulted in possibilities of health beneficial effects associated with their utilization. For example, there have been studies conducted which reported free radical scavenging and anti-proliferative capacities of pumpkin and parsley seed flour extracts. Also, recently, it was learned that carrot, cucumber, and broccoli seed flours have effects on GUT microbiota profile alteration, free radical scavenging, and anti-inflammatory capacities.

Tomato is the second most consumed vegetable in the world, and often processed into ketchup, tomato paste or juice. During tomato processing, 5-15% of the pre-processed material is left as waste. Seeds constitute 40-48% of this remaining waste product.

In recent years, the use of natural food by-products has been increasing with increasing interest in sustainability. This effort can reduce environmental contamination and at the same time, may add value to the final food products since natural food by-products are often rich in bioactive compounds. The tomato seed oil was found to be rich in lycopene and tocopherols, and tomato skin is rich in essential amino acids and polyphenolic compounds.

In addition, tomato seed oil and skin have been evaluated for their potential health beneficial properties, such as antioxidant activities of tomato skin extracts, as well as defatted seed's lipoprotein-lowering effects in blood and liver. Because of bioactive content and health beneficial properties, tomato skin and seed oil are currently utilized as salad topping and dressing, respectively.

However, tomato seed flour, which is a by-product from tomato seed oil, is still treated as waste. Tomato seed flour may possess more health beneficial properties which were worth of further study. In addition, there is a lack of information on the bioactive components which were responsible for the bioactivities in tomato seed flour, warranting additional research to reveal.

It would be highly desirable to evaluate tomato seed flour for its health beneficial effects and potentials for use as the GUT microbiota profile modulator.

Probiotics are defined as “live micro-organisms which confer a health benefit on the host when administered in adequate amounts. Examples of the well known probiotic foods are yogurt, pickles, miso soup, and kombucha tea.

On the other hand, prebiotics are not widely known. Prebiotics may be defined as a non-digestible food ingredient that beneficially affects a host by selectively stimulating the growth and/or the activity of one or a limited number of bacteria in the colon. Because prebiotics are not live micro-organisms, prebiotics are not affected by heat, cold, acid, and time. Therefore, prebiotics may be much more efficient, than probiotics, in terms of altering the GUT microbiota profile.

Accumulated data suggest that prebiotics, particularly macronutrients, play a major role in shaping the composition and activity of GUT microbiota populations. Several studies have linked the microbial metabolism of macronutrients such as anthrocyanins and polyphenols to chronic diseases such as obesity, celiac disease, cardiovascular disease, type 2 diabetes, and cancers.

Vegetable seeds, including tomato seeds, are one of the major byproducts from the manufacturer of vegetable juice. Even though seeds are byproducts, they contain a high concentration of macronutrients. Therefore, by validating vegetables, such as tomato, seeds' biological effects, the agricultural industry can add value to their byproducts and further, such as tomato, seeds' biological effects, the agricultural industry can add value to their byproducts, and further process seeds into seed oils and flours. These new products may then be used as prebiotics and value-added food products, as described in the present invention.

It would be highly desirable to provide a method for modulating microbiota in the gastrointestinal tract (GUT) of a user based on evaluation and validation of the biological effects of the tomato seed flour and oil on the health state of the user.

### SUMMARY OF THE INVENTION

It is, therefore, an object of the present invention to modulate the gastrointestinal tract (GUT) microbiota profile for improving the health of a user using seed powder/meal and/or seed oil and/or seed flour derived by processing byproducts of plants such as fruits, vegetables, and berries to reduce oxidative stress and inflammation, as well as to reduce the risk of obesity, diabetes, and other chronic diseases.

It is another object of the present invention to provide a method that involves the consumption of a tomato seed powder/meal, and/or tomato seed oil, and/or tomato seed flour, or a blend of these ingredients, or a product containing any of these ingredients, or an extract of any of these ingredients, which may be used as nutraceuticals, or bioactive food ingredients, or functional foods, or dietary supplements, commonly named as a food additive.

It is also an object of the present invention to provide the tomato seed powder/meal/flour/oil as a prebiotic source which may result in disease prevention and health promotion.

A further object of the present invention is to provide a method for modulating microbiota profile in the GUT of a user by processing seeds of a plant (such as fruits, vegetables, berries) to produce seed derivatives selected from a group including a seed oil, a seed flour, and combinations thereof, where the seed derivative has high Total Phenolic Content and sufficient free radicals scavenging and anti-inflammation capacities.

An additional object of the present invention is to provide a method for validating biological effects of tomato seed flour and/or tomato seed oil the GUT microbiota profile and evaluating health benefits provided by increased total phenolic content, free radicals scavenging, and anti-inflammation capacity of the tomato seed flour and/or oil.

In one aspect, the present invention is directed to a method for modulating microbiota in the gastrointestinal tract (GUT) of a user. The subject method assumes the steps of:

processing seeds of at least one of fruits, vegetables, berries, and their combination to produce a seed derivative such as a seed powder/meal, and/or a seed oil, and/or a seed flour, their extracts, and their combinations, and

validating biological effects of the seed derivative for ability to modulate the GUT microbiota, a Total Phenolic Content, and free radical scavenging and anti-inflammation capacities.

The method further includes the step of consuming the seed derivative by the user to result in modulating the GUT microbiota profile to attain reduction of oxidative stress, inflammation, and risk of chronic diseases through the interaction of the phenolic content and free radical scavenging and anti-inflammatory capacities of the seed derivative with the GUT microbiota.

In one exemplary implementation, the plant is a tomato, and the seed derivative is either the tomato seed flour, the tomato seed oil, tomato seed powder/meal, or their combinations, or their extracts.

In the subject method, for validating the biological effects of the seed derivative consumption, a tomato seed flour sample extract was prepared by:

weighting a tomato seed flour sample of 10 g of tomato seed flour, and

extracting the tomato seed flour sample extract three consecutive times, each extraction with 25 mL of 50% acetone. Subsequently, the tomato seed flour sample extract was studied for biological effects on the GUT microbiota. In addition, alternative solvents, including ethanol/water and acetone/water at ratios ranging from 100:0 to 0:100 (v/v) may be used to prepare the seed derivatives extracts using reflux, percolation, soaking with or without heat and/or microwaving, and Soxhlet extraction methods, and followed by removing the solvent(s) and water.

In the subject method, two separate batches of tomato seed flours were extracted, and analyzed for their chemical compositions, total phenolic content, and potential health benefits, particularly free radical scavenging capacities, anti-inflammatory capacities, and gut microbiota profile modulation. The findings could serve as a scientific basis for the development of food products using tomato seed flours to improve human health, as well as further investigation of the biological benefits and molecular mechanisms behind it.

For the GUT microbiota profile alteration, the GUT microbiota complex was prepared which contained Bacteroidetes and Firmicutes phyla, and Akkermansia, Bifidobacteria, Enterobacteriaceae, Lactobacillus, Prevotella and Ruminococcus genera, and the biological effects of the tomato seed flour on the GUT microbiota was validated by applying the 16S rRNA gene sequencing to Bacteroidetes and Firmicutes phyla, and Akkermansia, Bifidobacteria, Enterobacteriaceae, Lactobacillus, Prevotella and Ruminococcus genera in the GUT microbiota complex reacted with the tomato seed flour sample extract.

The 16S rRNA gene sequencing preferably includes the steps of: treating the GUT microbiota complex with 0.1% of the tomato seed flour sample extract,

extracting bacterial DNA from the GUT microbiota complex treated with the tomato seed flour sample extract,

performing Real-Time Polymerase Chain Reaction (PCR) with a reaction system containing 10 μL SYBR®Green Real-SCR Master Mix, 0.25 μL 500 nM oligo primers, 4.5 μL water, and 5 μL of said bacterial DNA, and

determining a relative content of the Bacteroidetes and Firmicutes phyla, and Akkermansia, Bifidobacteria, Enterobacteriaceae, Lactobacillus, Prevotella and Ruminococcus genera in the reaction system.

In addition, the subject method includes the step of validating the biological effects by measuring Total Phenolic Content (TPC) of the tomato seed flour sample extract by:

analyzing the TPC of a reaction mixture of the tomato seed flower sample extract and gallic acid by the Folin-Ciocalten colorimetric method,

measuring the absorbance of the reaction mixture of the tomato seed flour sample extract and gallic acid at 765 nm. The TPC is expressed as mg gallic acid equivalent (GAE) per gram of the tomato seed flow sample extract. The TPC of the tomato seed flour sample extract is 1.97-2.00 mg gallic acid equivalent/g (GAE/g).

Furthermore, the subject method validates the biological effects for the free radical scavenging and anti-inflammatory capacities of the tomato seed flour sample extract. In order to validate the free radical scavenging capacity, a method is selected from a group comprising: oxygen absorbing capacity (ORAC), relative 2,2-diphenyl-1-picrylhydrazyl (DPPH) radical scavenging capacity (RDSC), ABTS⋅+ scavenging capacity, and their combination.

The subject method also validates the anti-inflammatory capacity by studying the inflammatory response of interleukin-beta (IL-113), interleukin-6 (IL-6), and tumor necrosis factor alpha (TNF-d) inflammation markers reacted with the tomato feed flour sample extract.

Furthermore, the subject method validates the GUT microbiota profile modulating capacity of the tomato seed oil and compares with the GUT microbiota profile modulation capacity of the tomato seed flour. For the study of the tomato seed oil's GUT microbiota profile modulating capacity, a tomato seed oil sample extract was prepared. In addition, the GUT microbiota complex was prepared which contained Bacteroidetes and Firmicutes phyla, and Akkermansia, Bifidobacteria, Enterobacteriaceae, Lactobacillus, Prevotella and Ruminococcus genera.

Subsequently, the biological effects of the tomato seed oil sample extract on the GUT microbiota were studied by applying the 16S rRNA gene sequencing to Bacteroidetes and Firmicutes phyla, and Akkermansia, Bifidobacteria, Enterobacteriaceae, Lactobacillus, Prevotella and Ruminococcus genera in the GUT microbiota complex reacted with the tomato seed oil sample extract.

The validation of the biological effects of the seed derivatives resulted in a detection of a significant increase of the ratio between Bacteroidetes and Firmicutes phyla. This finding proves the potential of consumption of the tomato seed flour in controlling body weight gain and reducing the risk of obese-related chronic diseases.

Also, an increase of Bacteroidetes phylum, and a decrease of Firmicutes phylum have been detected, thus proving a potential of consumption of the tomato seed flour in health-beneficial effects related to nutrition, xenobiotic, drug metabolism, antimicrobial protection, and immune enhancement.

In addition, an increase in the Akkermansia genus has been detected which proves potential of consumption of the tomato seed flour in reduction of the risk of developing obesity and type 2 diabetes and the increasing possibility of reversing obesity and type 2 diabetes.

Furthermore, the following GUT microbiota profile alterations have been detected:

reduction of Bifidobacteria genus and increase of Lactobacillus genus have been detected proving a potential of consumption of the tomato seed flow in preventing infectious diarrhea, carcinogenic activity, and treating lactic acidosis,

reduction in Enterobacteriaceae genus proving a potential of consumption of tomato seed flour in reduction of pro-inflammatory pathobionts, and

reduction in Prevotella genus and increase in Ruminococcus genus proving a potential of consumption of tomato seed flow in lowering the risk of chronic inflammatory disease.

Also, the subject method's validation of the anti-inflammatory capacity of the tomato seed flour includes detection of suppression of mRNA-expressions of the pro-inflammation genes including IL-1β, IL-6, and TNF-α, thus proving a potential of consumption of the tomato seed flour in treating inflammation and inflammation related chronic diseases.

The subject method's step of validation of the free radicals scavenging capacities of the tomato seed flour sample extract was conducted against ORAC, DPPH and ABTS assays which resulted in the levels of 86.3-88.6, 3.6-3.8 and 3.4-3.6 μmoles (TE)/g, respectively, thus proving a potential of the consumption of the tomato seed flour in scavenging free radicals.

In another aspect, the present invention addresses a seed-based food additive for modulating microbiota in the gastro-intestinal tract (GUT) of a user. The subject seed-base food additive constitutes a seed derivative selected from a group including a seed oil and/or a seed flour and/or seed powder/meal, and their extracts, prepared by processing of at least one plant selected from a group of fruits, vegetables, berries, and their combination. The subject seed derivative is characterized by:
(a) an increased Total Phenolic Content (TPC) ranging from 1.97 to 2.00 mg GAE/g beneficial in free radicals scavenging capacity of the seed derivative,
(b) an ability to increase a ratio between Bacteroidetes and Firmicutes phyla of the GUT microbiota beneficial in controlling body weight gain and reducing the risk of obese-related chronic diseases, and
(c) an ability to increase Bacteroidetes phylum and to decrease Firmicutes phylum, beneficial in promoting health-beneficial effects related to nutrition, xenobiotic, drug metabolism, antimicrobial protection, and immune enhancement.

The seed-based food additive is further characterized by an ability to:

increase Akkermansia genus in the GUT microbiota to reduce the risk of obesity and type 2 diabetes,

to reduce Bifidobacteria genus and increase Lactobacillus genus to prevent infectious diarrhea and carcinogenic activity, and to treat lactic acidosis,

to reduce Enterobacteriaceae genus in the GUT microbiota to reduce pro-inflammatory pathobionts,

to reduce Prevotella genus and to increase Ruminococcus genus in the GUT microbiota to prevent a risk of chronic inflammatory disease, to suppress pro-inflammatory genes, and to treat inflammation related chronic diseases,

where the seed-based food additive has free radicals scavenging capacity evaluated against ORAC, DPPH and ABTS assays of the levels of 86.3-88.6, 3.6-3.8 and 3.4-3.6 μmoles (TE)/g, respectively.

These and other objects and advantages of the present invention will be apparent in view of the further Drawings and description of the preferred embodiment of the present invention.

### BRIEF DESCRIPTION OF THE DRAWINGS

FIG. 1 represents a scheme for the tomato purée and paste processing, and depicts schematically processing of the by-products, such as tomato seeds, into the tomato seed oil and flour;

FIG. 2 is representative of changes in the GUT microbiota profile detected by the Bacteroidetes/Firmicutes phyla ratio;

FIGS. 3A-3B show changes in the GUT microbiota profile detected for Bacteroidetes phyla (FIG. 3A) and Firmicutes phyla (FIG. 3B), respectively;

FIG. 4 is a representative of the changes in the GUT microbiota profile detected for the Akkermansia genus;

FIGS. 5A-5B are representatives of the changes in the GUT microbiota profile detected for Bifidobacteria genus (FIG. 5A) and Lactobacillus genus (FIG. 5B), respectively;

FIG. 6 is representative of the changes in the GUT microbiota profile detected for the Enterobacteriaceae genus;

FIGS. 7A and 7B are representative of the changes in the GUT microbiota profile for Prevotella genus (FIG. 7A) and Ruminococcus genus (FIG. 7B), respectively;

FIG. 8 is representative of the Total Phenolic Content of the tomato seed flour extract;

FIGS. 9A-9F are representative of concentration dependent anti-inflammatory capacities of the tomato seed flour extract for IL-1α (FIGS. 9A-9B), IL-6 (FIGS. 9C-9D), and IL-β (FIGS. 9E-9F), respectively, for the TFO (FIGS. 9A, 9C, 9E) and for the TFN (FIGS. 9B, 9D, 9E), respectively;

FIGS. 10A-10B are representative of changes in the GUT microbiota profile detected for Bacteroidetes/Firmicutes phyla ratio for tomato seed flour (FIG. 10A) and tomato seed oil (FIG. 10B);

FIGS. 11A-11B are representative of the changes in the GUT microbiota profile detected for the Akkermansia genus for tomato seed flour (FIG. 11A) and tomato seed oil (FIG. 111B), respectively;

FIGS. 12A-12D are representative of the changes in the GUT microbiota profile detected for Bifidobacteria genus (FIGS. 12A-12B), Lactobacillus genus (FIGS. 12C-12D) for the tomato seed flour (FIGS. 12A, 12C) and for the tomato seed oil (FIGS. 12B, 12D), respectively;

FIGS. 13A-13B are representative of the changes in the GUT microbiota profile detected for Enterobacteriaceae genus for the tomato seed flour (FIG. 13A) and the tomato seed oil (FIG. 13B), respectively;

FIGS. 14A-14C are diagrams of a typical UHPLC-PDA chromatogram (FIG. 14A) and total ion current (TIC) chromatogram (FIG. 14B) of tomato seed flour extract with FIGS. 14C and 14D being the diagrams of MS and MS/MS spectra, respectively, of peak 3 at the retention time of 18.44 min;

FIGS. 15A-15F are representative of the anti-inflammatory capacities of tomato seed flour extracts in THP-1 macrophages, with FIGS. 15A, 15C, 15E for the first batch of tomato seed flour extract TSF1, and FIGS. 15B, 15D, 15F for the second batch of tomato seed flour extract TSF2, respectively; and

FIGS. 16A-16H are diagrams representative of the GUT microbiota profile modulation caused by tomato seed flour extracts, for Bacteroidetes phylum (FIG. 16A), Firmicutes phylum (FIG. 16B), Akkermansia genus (FIG. 16C), Bifidobacterium genus (FIG. 16D), Lactobacillus genus (FIG. 16E), Enterobacteriaceae genus (FIG. 16F), Prevotella genus (FIG. 16G), and Ruminococcus genus (FIG. 16H).

### DETAILED DESCRIPTION OF THE PREFERRED EMBODIMENT

The present invention relates generally to the use of seed powder/meals, flours and oils, as well as their extracts, to improve a user's health including GUT microbiota profile modulation with the purpose of reducing oxidative stress and inflammation, as well as to reduce the risk of obesity and other chronic diseases.

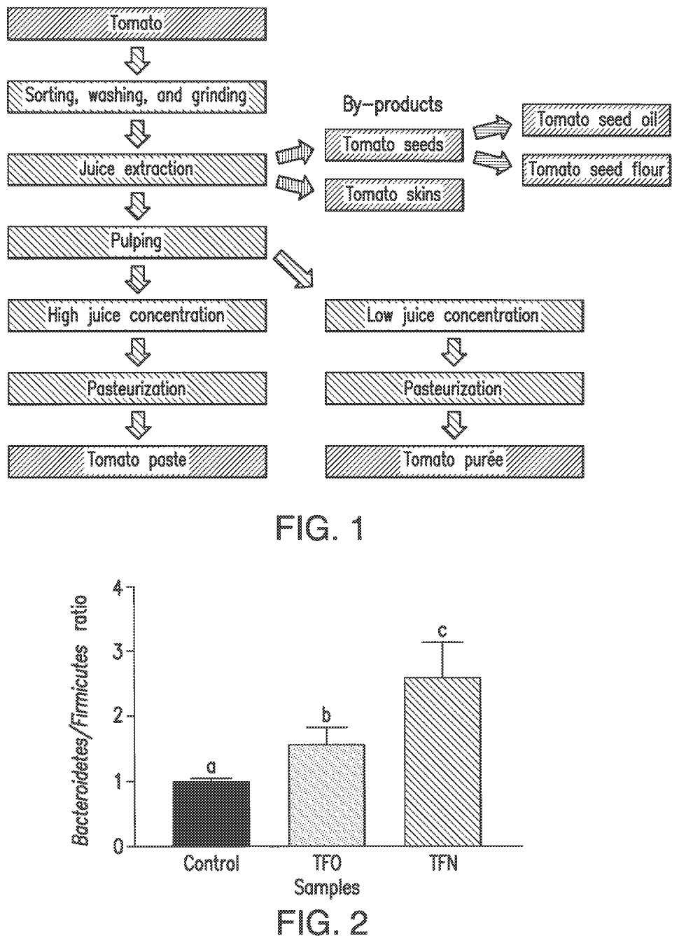

Although seeds of numerous plants, such as vegetables, fruits, and berries, singularly or in combination, may be candidates for alteration of GUT microbiota profile as an important factor for human health, seeds of tomatoes will be further exemplified as one of the fruits, vegetable and berries representative as a source for production of the health beneficial food additive. As an example, but not to limit the scope of the present invention to a single particular implementation, tomato seed flour and tomato seed oil will be further addressed in the following paragraphs. FIG. 1 represents a scheme for the tomato purée and paste processing, and depicts schematically processing of the by-products, such as tomato seeds, into the tomato seed oil and tomato seed flour;

Tomato seed flour and tomato seed oil have been investigated herein for their biological effects on the GUT microbiota profile and were observed to effectively modulate the GUT microbiota profile, through their increased Total Phenolic Content, free radial scavenging and anti-inflammatory capacitance.

The tomato seed flour/oil has also been observed in the subject studies to alter the GUT microbiota profile as the source of pre-biotic and its effect in disease prevention and health promotion.

In addition, the tomato seed flour and/or oil and their extracts have been observed in the present studies to significantly increase the relative abundance of the Akkermansia genus. This level has been inversely correlated with body weight in rodents and humans.

As a result, tomato seed flour and/or oil, and their extracts, are promising candidates in GUT microbiota profile alteration, and provide dietary phenolics and free radical scavenging and anti-inflammatory components. Therefore, tomato seed flour and oil, and their extracts, as a result of the subject method validation of biological effects of the tomato seed flour and oil, may be categorized as having a high potential for being used as nutraceuticals in functional food and dietary supplement products for disease prevention and health promotion.

GUT microbiota profile is an important factor for human health or disease. Each bacteria phylum or genus has its own role in the human body system. Therefore, maintaining health through GUT microbiota profile by consuming healthy foods (probiotics or prebiotics) is important. In the present invention, tomato seed flour/oil showed promising results in GUT microbiota profile operation, and reducing body weight.

Specifically, in order to validate the biological effects of the tomato seed flour, 16S rRNA gene sequencing was used in the present method. 16S rRNA gene sequencing has been chosen in the present method as a preferred method in microbiota research for the following reasons: (a) it is present in almost all bacteria, often existing as a multi gene family, or operons; (b) the function of the 16S rRNA gene does not change over time; and (c) the 16S rRNA gene (1,500 bp) is large enough for informatics purposes.

After choosing the method (16S rRNA sequencing), specific phyla and genera have selected for the subject method. In total, eight phyla and genera have been tested which include Bacteroidetes and Firmicutes phyla, and Akkermansia, Bifidobacteria, Enterobacteriaceae, Lactobacillus, Prevotella, and Ruminococcus genera, with the functions closely related to human health.

Also, tomato seed flour extracts were produced and their Total Phenolic Contents (TPC) have been measured. Phenolic compounds are often found in healthy food such as fruits and vegetables. Health promoting effects of phenolic compounds on the human body include scavenging of free radicals generated inside the human body. In addition, some phenolic compounds can interact with the GUT microbiota. Therefore, evaluating the concentration of phenolic compounds in food is essential for validating its biological effects on the health state.

In addition to the Total Phenolic Content of the tomato seed flour, free radicals scavenging and anti-inflammatory capacities have been evaluated using tomato seed flour extracts. These two assays are closely related to human chronic diseases. For example, excessive free radicals trigger pro-inflammatory responses and may cause various chronic diseases including obesity, celiac disease, cardiovascular disease, type 2 diabetes and cancers. Therefore, evaluating free radicals scavenging and anti-inflammatory capacities are important for chronic disease prevention.

For validating free radicals scavenging capacities of the tomato seed flour, the present method included three different routines, such as: (a) oxygen radical absorbing capacity (ORAC), (b) relative 2,2-diphenyl-1-picrylhydrazyl (DPPH) radical scavenging capacity (RDSC), and (c) ABTS+ scavenging capacity.

For the anti-inflammatory capacity validation of the tomato seed flour, three pro-inflammatory genes have been chosen as inflammation markers, including interleukin-1 beta (IL-1β), interleukin-6 (IL-6), and tumor necrosis factor alpha (TNF-α)

The subject method has been developed to validate the efficacy of an exemplary embodiment of the invention (such as the tomato seed flour and tomato seed oil) as candidates for the GUT microbiota profile alteration, as will be detailed in the following paragraphs.

Tomato Seed Flour

The subject method, in its initial step, assumes the preparation of the tomato seed flour sample extract. For this, 10 grams of tomato seed flour samples were accurately weighed, and subsequently extracted three consecutive times with 25-50 mL of 50% acetone.

In addition, alternative solvents, including ethanol/water and acetone/water at ratios ranging from 100:0 to 0:100 (v/v) may be used to prepare the seed derivatives extracts using reflux, percolation, soaking and/or Soxhlet extraction methods, with the subsequent removal of the solvent(s) and water.

The tomato seed flour samples were obtained from two different batches (TFO from an old batch, and TFN from a new batch). The old batch relates to tomato seed flour which was produced a predetermined time period prior to the tomato seed flour sample from the new batch. All experiments were performed in triplicate.

Subsequently, 16S rRNA gene sequencing has been applied to the tomato seed flour sample extracts both for the TFO and TFN. In this routine, the GUT microbiota complex was treated with and without 0.1% of sample extracts. Bacterial DNA was extracted with QIAamp DNA MiniKit following the manufacturer's protocol. Real-Time PCR (Polymer Chain Reaction) was performed with a reaction system of 10 μL SYBR®Green Real-Time PCR Master Mix, 0.25 μL 500 nM custom-made oligo primers, 4.5 μL water, and 5 μL DNA. Primers specific for Bacteroidetes and Firmicutes phyla and Akkermansia, Bifidobacteria, Enterobacteriaceae, Lactobacillus, Prevotella, and Ruminococcus genera were used to determine the relative abundance of respective microorganisms.

Furthermore, the Total Phenolic Content (TPC) of the tomato seed flour sample extracts (for TFN and TFO) was analyzed by the Folin-Ciocalteu colorimetric method using gallic acid as a standard. Tomato mixtures were prepared by mixing the tomato seed flour sample extracts (for TFN and TFO) with gallic acid. After 2 h of reaction of the tomato seed flour sample extracts with gallic acid at ambient temperature, the absorbance of each reaction mixture was measured at 765 nm. TPC was expressed as mg gallic acid equivalents (GAE) per gram of tomato seed flour.

In addition, the free radicals scavenging capacities were measured against several assays. The oxygen radical absorbing capacity (ORAC) values were measured according to a laboratory original protocol with a modification which used a Victor3 multi-label plate reader (PerkinElmer, Turku, Finland). In the subject method, the final reaction mixture consisted of 225 μL of 8.16×10-8 M FL, 30 μL of sample or solvent blank or standard, and 25 μL of 0.36 M AAPH. The fluorescence of the final reaction mixture was recorded every 2 min over 2 h at 37° C. Excitation and emission wavelengths were 485 and 520 nm, respectively. Trolox was used as a standard, and the results were reported as μmol TE/g tomato seed flour.

The radical scavenging capacity (RDSC) was evaluated according to a laboratory method. Sample extracts, Trolox standards, or blank solvent control were added to 0.1 mL of freshly prepared 2,2-diphenyl-1-picrylhydrazyl (DPPH) solution to initiate the reaction. The absorbance of the reaction mixture was measured at 515 nm every minute for 40 min of reaction in dark. DPPH scavenging capacities were calculated using the areas under the curve and expressed as micromoles of Trolox equivalents (TE) per gram of tomato seed flour.

Subsequently, the scavenging ability against ABTS⋅+ was measured. The ABTS⋅+ working solution was prepared by reacting ABTS with manganese oxide and diluting to an absorbance of 0.700±0.005 at 734 nm. The final reaction mixture consisted of 80 μL sample or solvent or standard, and 1 mL ABTS⋅+ working solution. After being vortexed for 30 s, the absorbance was read at 734 nm after 90 s of reaction. Trolox was used as a standard. The results were expressed as micromoles of TE/g of tomato seed flour. Each flour extract was measured in triplicate.

For measuring the anti-inflammatory capacity, THP-1/PMA macrophage cells (5×105 cells/mL) were cultured in RPMI1640 with 10% FBS and 1% penicillin and streptomycin at 37° C. under 5% CO2 in six-well plates to reach an 80% confluence for 48 hours. After 48 hours, cells were incubated with tomato seed flour sample extract for 24 hours. The medium was changed every 24 hours.

After 4 hours of induction with 10 ng/mL lipopolysaccharide (LPS), the culture medium was discarded, and the cells were collected for RNA isolation and real-time PCR analysis.

cDNA synthesis kit was used to reverse transcribe cDNA. Real-time PCR was performed on a ViiA7 Sequence Detection System using TaqMan Universal PCR Master Mix. IL-1β, IL-6 and TNF-α primers were used for inflammatory response and TBP (TATA binding protein) was used for the control.

The following amplification parameters were used for PCR: 50° C. for 2 min, 95° C. for 10 min, with 46 cycles of amplification at 95° C. for 15 s and 60° C. for 1 min.

Statistical analysis of the obtained results for the biological effects validation of the tomato seed flour used means±standard deviation (SD) for each data point. For comparison, a t-test or one-way analysis of variation (ANOVA) (P≤0.05) followed by a post hoc test (Tukey test) was used.

The results of the validation of the biological effects of the tomato seed flour with regard to the GUT microbiota alteration are presented in the following paragraphs.

The GUT profile alteration with regard to the Bacteroidetes/Firmicutes ratio is presented in FIG. 2, where TFO presents the old batch of tomato seed flour, TFN presents the new batch of tomato seed flour, letters (a,b,c) indicate a statistically significant difference (P≤0.05).

The ratio between Bacteroidetes and Firmicutes is closely related to obesity. Both TFO and TNF showed a significant increase of the Bacteroidetes/Firmicutes ratio, suggesting the potential of tomato seed flour in controlling body weight gain and reducing the risk of obese-related chronic diseases such as diabetes.

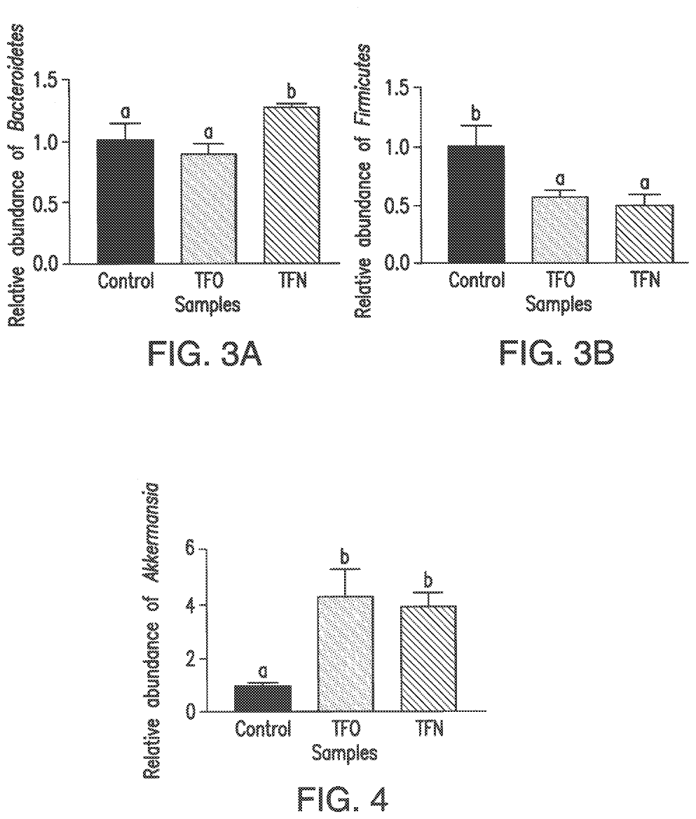

The GUT profile modification with regard to Bacteroidetes and Firmicutes phyla is presented in FIGS. 3A and 3B, respectively, where TFO presents the old batch of tomato seed flour, TFN presents the new batch of tomato seed flour, and the letters (a and b) indicate a statistically significant difference (P≤0.05).

As shown in FIG. 3A, the TFO extract did not result in any changes to the Bacteroidetes phylum. On the other hand, the TFN extract increased the relative abundance of the Bacteroidetes phylum. In FIG. 3B, regarding the Firmicutes phylum, both TFO and TFN extracts significantly reduced the relative abundance of Firmicutes phylum. Low-calorie diets through either fat or carbohydrate restriction are known to result in an increase in Bacteroidetes and the reduction of Firmicutes. Thus, the detected alteration of the GUT microbiota shown in FIGS. 3A-3B has potential health-beneficial effects. Both Bacteroidetes and Firmicutes phyla abundance alteration suggest that consumption of tomato seed flour and components may have potential health-beneficial effects related to nutrition, xenobiotic and drug metabolism, antimicrobial protection, and immune enhancement.

As shown in FIG. 4, in the present example embodiment, both TFO and TFN significantly increased the relative abundance of the Akkermansia genus.

Akkermansia muciniphila is a mucin-degrading bacterium that resides in the mucus layer and belongs to Akkermansia genus. The presence of this bacterium inversely correlates with body weight in rodents and humans. The Akkermansia muciniphila is known also to decrease obesity and type 2 diabetic in mice. So, increase in the abundance of the Akkermansia genus resulted from the tomato seed flour may benefit those who are obese or have high risk of obesity and/or type 2 diabetes. In the present invention, as depicted in FIG. 4, both TFO and TFN showed a significant increase of the Akkermansia genus. Therefore, consumption of tomato seed flour may reduce the risk of or reverse obesity and type 2 diabetes by altering the Akkermansia population in the GUT.

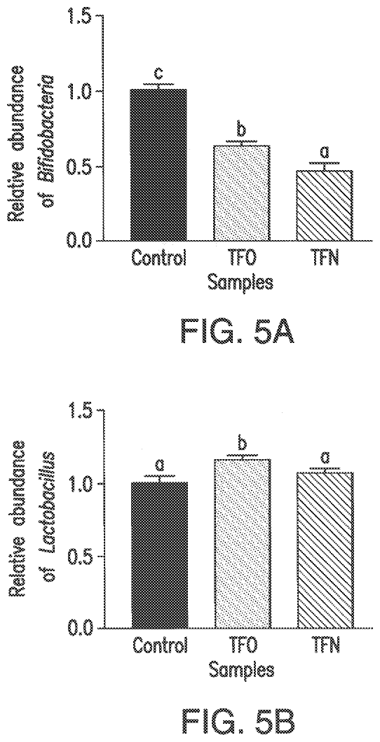

The changes in Bifidobacteria and Lactobacillus genera population in the GUT, resulted from reaction with tomato seed flour sample extract, are shown in FIGS. 5A-5B, respectively. FIG. 5A depicts a significant reduction of Bifidobacteria.

For Lactobacillus, as presented in FIG. 5B, the TFO treatment resulted in an increase of Lactobacillus. However, no significant difference was observed in the TFN treatment.

Bifidobacteria and Lactobacillus genera are probiotic bacteria. Maintaining a certain amount of these bacteria has several health beneficial effects due to their functions. For example, Bifidobacteria and Lactobacillus are able to prevent or alleviate infectious diarrhea through their effects on the immune system and resistance to colonization by pathogens. Also, there is some experimental evidence that certain Bifidobacteria may actually protect the host from the carcinogenic activity of intestinal flora.

However, Bifidobacteria and Lactobacillus are the major bacteria that produce lactic acid. Overabundance of these genera can cause lactic acidosis. To have health benefits and prevent lactic acidosis, maintaining certain levels of the Bifidobacteria and Lactobacillus genera is crucial. Since both TFO and TFN reduced the relative abundance of the Bifidobacteria, these samples may be used to treat or prevent lactic acidosis.

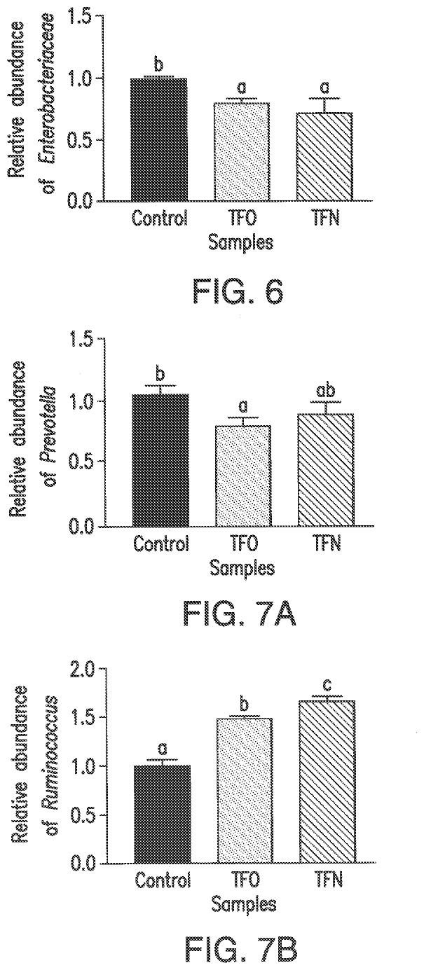

FIG. 6 depicts changes in the GUT microbiota profile regarding the Enterobacteriaceae genus. The Enterobacteriaceae genus is known as a “bad” bacteria for being pro-inflammatory pathobionts. Therefore, reducing the Enterobacteriaceae's population may have various health beneficial effects. In the present method, both TFO and TFN extracts showed a significant reduction in the Enterobacteriaceae genus. This result suggests that tomato seed flour may promote the GUT health by reducing the Enterobacteriaceae genus.

FIGS. 7A-7B depict changes in the GUT microbiota profile regarding Prevotella genus (shown in FIG. 7A), and Ruminococcus genus (shown in FIG. 7B). Both TFO and TFN extracts were able to reduce the abundance of the Prevotella genus. On the other hand, both TFO and TFN significantly increased the relative abundance of the Ruminococcus genus.

It has been reported that long-term consumption of carbohydrates, especially fiber, has been linked to the increased relative abundance of the Prevotella genus. Together, the present results suggest potential health benefits of tomato seed flour intake.

Also, the Prevotella genus has been associated with chronic inflammatory disease through immune responses. While the intake of fiber is associated with increased relative abundance of the Prevotella genus, western diet, typically constituted by high consumption of red meat, animal fat, high sugar and low fiber was associated with an increased relative abundance of Ruminococcus genera. In the current method, the results presented in FIGS. 6A-6B suggest that tomato seed flour extract may lower the risk of chronic inflamatory disease through Prevotella genus-mediated improvement of immune responses.

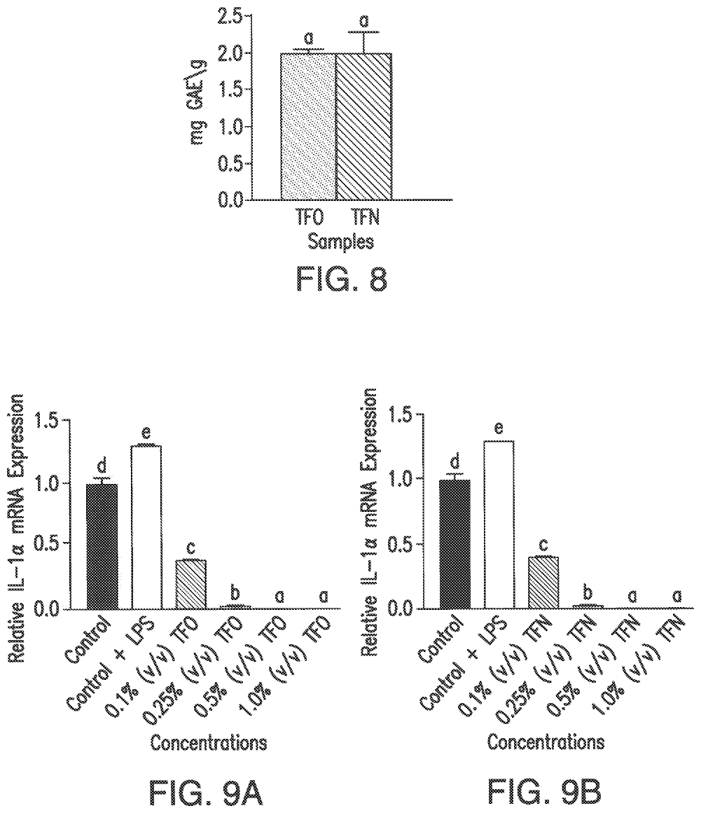

FIG. 8 depicts the results of the measurements of the Total phenolic content (TPC) of tomato seed flour extracts. The GAE stands for gallic acid equivalent, the TFO presents the old batch of tomato seed flour, the; TFN presents the new batch of tomato seed flour. Each column in FIG. 8 represents the mean±SD (n=3). All experiments were carried out in triplicate.

In the present example embodiment, the TFO and TFN had total phenolic levels of 2.00 and 1.97 mg GAE/g, respectively. These numbers are comparable to pomegranate seed flour extracts' (50% acetone)'s total phenolic content values (1.3-2.2 mg GAE/g). Compared to pomegranate seed extracts, tomato seed flour extracts had very similar total phenolic contents, suggesting possible health beneficial effects and utilization of tomato seed flours.

Table 1 is representative of the Free radicals scavenging capacities of the tomato seed flour.

TABLE 1

| | ORAC | DPPH | ABTS |
|---|---|---|---|
| SAMPLE | μmoles TE/g | μmoles TE/g | μmoles TE/g |
| TFO | 88.57a ± 2.42 | 3.57a ± 0.09 | 3.39a ± 0.08 |
| TFN | 86.32a ± 7.01 | 3.81a ± 0.20 | 3.58a ± 0.61 |

In Table 1, ORAC indicates relative oxygen radical absorbance capacity, DPPH indicates relative DPPH⋅ scavenging capacity, ABTS indicates relative ABTS^{⋅+} scavenging capacity, TE stands for Trolox equivalents, the TFO represents the old batch of tomato seed flour, while the TFN represents the new batch of tomato seed flour. All experiments were carried out in triplicate and expressed as mean±SD (n=3).

In the current method, the tomato seed flour extracts showed ORAC, DPPH and ABTS values of 86.3-88.6, 3.6-3.8, and 3.4-3.6 μmoles TE/g, respectively.

ORAC values of tomato seed flour extracts were compared to ORAC values of broccoli, carrot and cucumber seed flour extracts' (50% acetone), i.e., 633.5, 143.9 and 28.6 μmoles TE/g, respectively. Compared to broccoli or carrot seed flour extracts, ORAC values of the tomato seed flour were lower. However, compared to cucumber seed flour, tomato seed flour extracts had much higher ORAC values.

Similarly, broccoli, carrot and cucumber seed flour extracts from the previous study showed DPPH values of 84.8, 16.0 and 2.6 μmoles TE/g, respectively. Compared to broccoli or carrot seed flour extracts, DPPH values of tomato seed flour extracts were lower. But, compared to cucumber seed flour extract, tomato seed flour showed much greater DPPH values.

On the other hand, ABTS values of tomato seed flours (3.4-3.6 μmoles TE/g) were lower compared to broccoli, carrot and cucumber seed flour extracts' ABTS values of 175.8, 250.0 and 6.8 μmoles TE/g, respectively.

This result suggests that tomato seed flours have the potential to scavenge free radicals and may promote human health by scavenging free radicals.

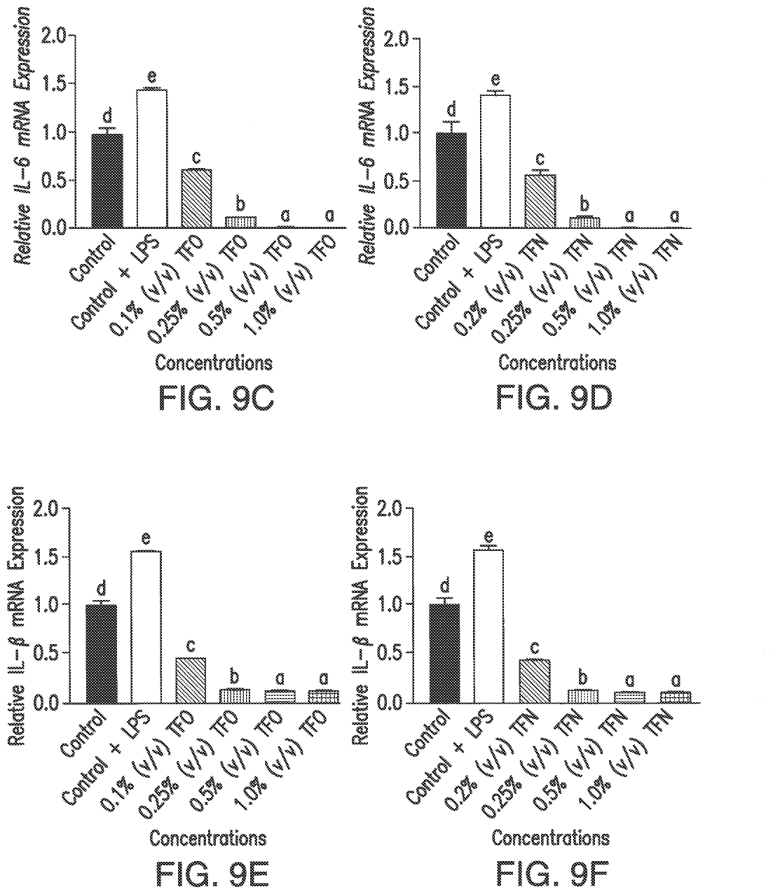

FIGS. 9A-8F depict concentration dependent anti-inflammatory capacities of tomato seed flour extracts for the pro-inflammatory genes IL-1β (FIG. 9A-9B), IL-6 (FIG. 9C-9D), and TNF-α (FIG. 9E-9F), respectively. All experiments were carried out in triplicate.

As shown in FIGS. 9A-9c, both TFO and TNO samples were able to suppress mRNA expressions of pro-inflammatory genes including IL-1β, IL-6 and TNF-α. Also, these inhibition effects were concentration dependent.

In the human body, inflammatory cells, such as macrophages, are known to produce pro-inflammatory cytokines at the site of inflammation. During the process, excessive pro-inflammatory cytokines production can cause oxidative stress and result in various chronic diseases. Therefore, inhibiting pro-inflammatory cytokines have been the target for the treatment and prevention of various chronic diseases.

The current results suggest that tomato seed flour extracts may be used as a food source to treat inflammation and inflammation related chronic diseases.

Summarizing the results presented in the previous paragraphs, it may be concluded that the tomato seed flour extracts were able to increase Akkermansia population and the ratio of Bacteroidetes/Firmicutes, along with alterations of other important GUT microbiota members under the in vitro experimental conditions. The tomato seed flour extracts also contained a significant level of natural phenolics and have significant antioxidants and anti-inflammatory activities. The results suggest the potential of tomato seed flour in controlling body weight gain, and reducing the risk of obese-related and oxidative stress-related, and/or inflammation-related chronic diseases.

Tomato Seed Oil

The present method, in addition to the tomato seed flour, also addresses the bio-effects of the tomato seed oil on the GUT microbiota profile.

In order to validate the biological effects of the tomato seed oil, 16S rRNA gene sequencing was chosen for a reason similar to those presented in previous paragraphs relative to the study of the tomato seed flour, which include the presence in almost all bacteria, often existing as a multi gene family, or operons, the function of the 16S rRNA gene does not change over time, and the 16S rRNA gene (1,500 bp) is large enough for informatics purposes. Six specific phyla and genera were selected for testing, including Bacteroidetes and Firmicutes phyla, and Akkermansia, Bifidobacteria, Enterobacteriaceae, and Lactobacillus genera, because functions of these phyla and genera are closely related to human health. Such functions are as follows:

(a) low-calorie diets through either fat or carbohydrate restriction resulted in an increase in Bacteroidetes;

(b) the reduction of Firmicutes has the potential to beneficially affect one's health;

(c) Akkermansia is related to energy metabolism and balance. Also, the Akkermansia population is negatively associated with the consumption of polysaccharides;

(d) Bifidobacteria population was shown to correlate positively with cholesterol intake and metabolism;

(e) Enterobacteriaceae genus is known as “bad” bacteria and recent studies suggest that soy-based diet can decrease the number of Enterobacteriaceae genus;

(f) a high-fat diet was shown to induce an increase in Lactobacillus, and Lactobacillus is also involved in simple sugar degradation;

(g) the ratio between Bacteroidetes and Firmicutes is closely related to obesity.

In the subject method's validation of the tomato seed oil, 10 grams of seed oil sample was accurately weighed, and extracted three consecutive times with 25 mL of 50% acetone (75 ml of 50% acetone in total) to prepare the tomato seed oil sample extract. All experiments were performed in triplicate.

Subsequently, 16S rRNA gene sequencing was applied to the GUT microbiota complex. For this step, the GUT microbiota complex was treated with and without 0.1% of the tomato seed oil sample extracts. Bacterial DNA was extracted with QIAamp DNA MiniKit following the manufacturer's protocol. Real-Time PCR was performed with a reaction system of 10 μL SYBR®Green Real-Time PCR Master Mix, 0.25 μL 500 nM custom-made oligo primers, 4.5 μL water and 5 μL DNA. Primers specific for Bacteroidetes, Firmicutes phyla, and Akkermansia, Bifidobacteria, Lactobacillus, Enterobacteriaceae genera were used to determine the relative abundance of respective microorganisms.

Means±standard deviation (SD) were used in the statistical analyses for each data point. For comparison, a one-way analysis of variation (ANOVA) (p 0.05) followed by a post hoc test (Tukey test) was used.

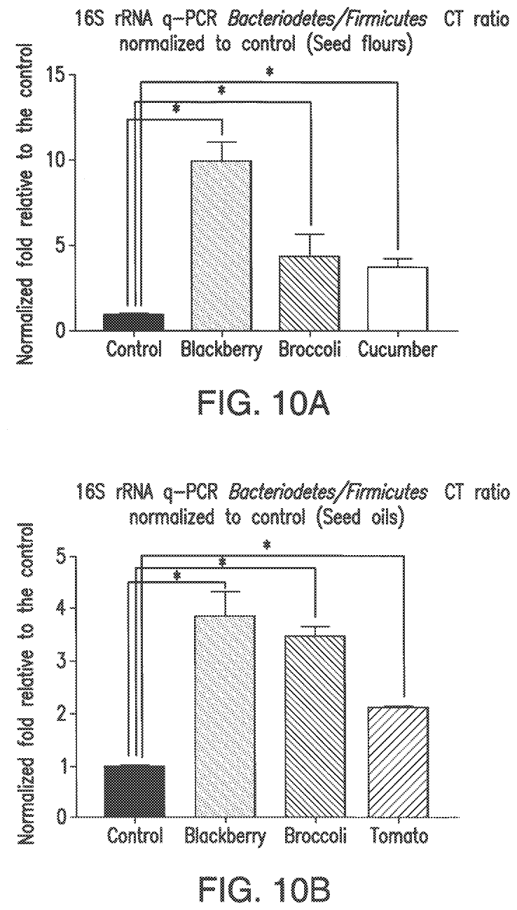

FIGS. 10A-10B depict the changes in gut microbiota profile for Bacteroidetes/Firmicutes ratio for the tomato seed flour samples (FIG. 10A) and tomato seedoil samples (FIG. 10B), where the asterisk indicates a statistically significant difference (* P≤0.05). Among various seed oils and flours, six samples in total showed a significant increase in the ratio of Bacteroidetes to Firmicutes.

The Bacteroidetes/Firmicutes ratio is closely related to obesity. Therefore, increasing this ratio may affect weight loss and reduce the risk of chronic diseases. Interestingly, within the six samples, blackberry seed flour, a byproduct of blackberry seed oil production, showed the most promising result.

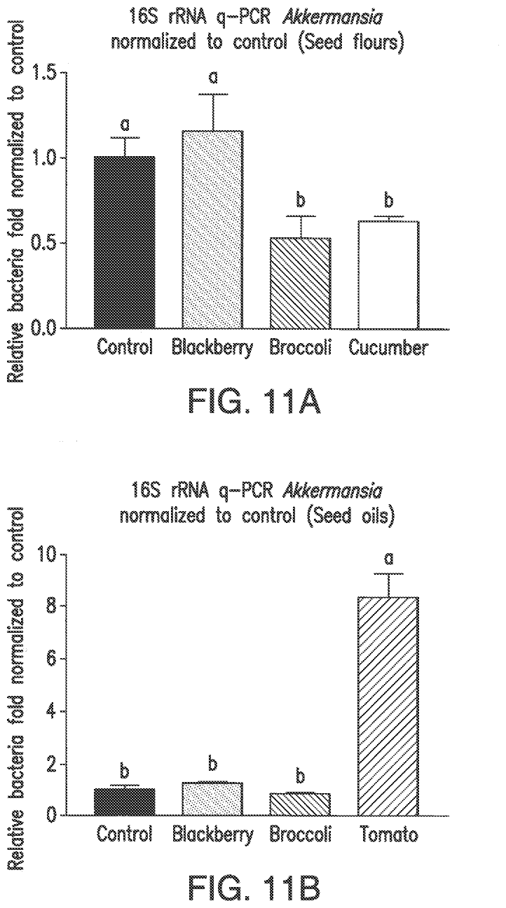

FIGS. 11A-11B depict changes in the GUT microbiota profile for Akkermansia genus for the tomato seed flour samples (FIG. 11A) the tomato seed oil samples (FIG. 11B).

Akkermansia muciniphila is a mucin-degrading bacterium that resides in the mucus layer and belongs to the Akkermansia genus. The presence of this bacterium inversely correlates with body weight in rodents and humans. Interestingly, it was found that the abundance of Akkermansia muciniphila decreased in obesity and type 2 diabetes in mice. Those individuals suffering from obesity or type 2 diabetes may need to increase the abundance of the Akkermansia genus. In the present example embodiment, tomato seed oil showed a sharp increase of the Akkermansia genus. Therefore, consumption of tomato seed oil may prevent or reverse obesity and type 2 diabetes by altering the Akkermansia population.

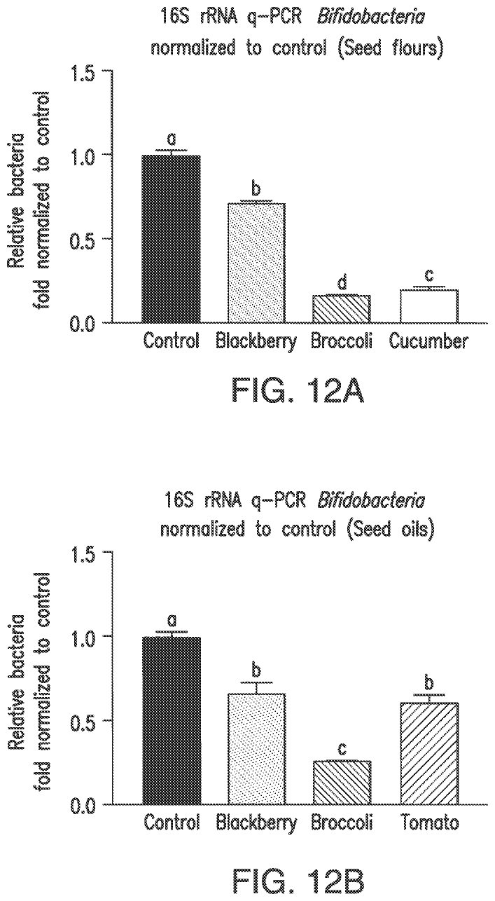

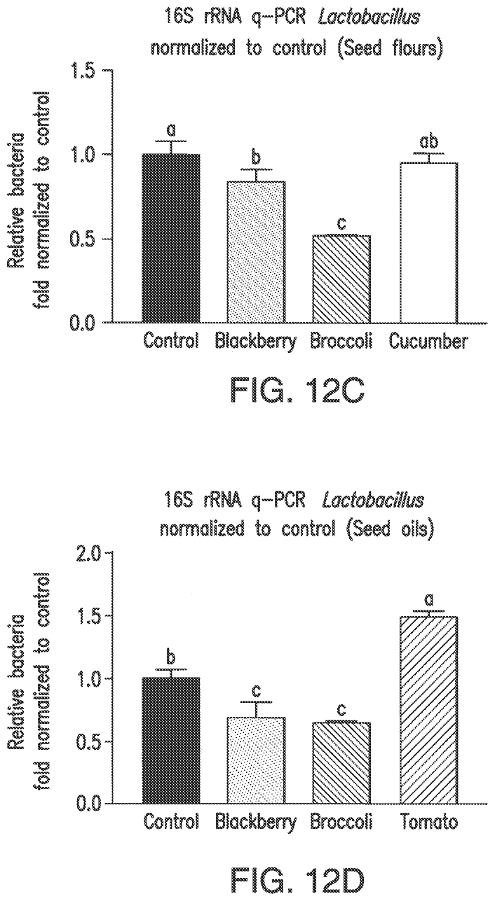

FIGS. 12A-12D depict the changes in the GUT microbiota profile for Bifidobacteria genus (FIGS. 12A-12B), Lactobacillus genus (FIGS. 12C-11D)), for tomato seed flour samples (FIGS. 12A, 12C) and tomato seed oil samples (FIGS. 12B, 12D).

Bifidobacteria and Lactobacillus genera are probiotic bacteria. Maintaining a certain amount of these bacteria has several beneficial health effects due to their functions. For example, Bifidobacteria and Lactobacillus are able to prevent or alleviate infectious diarrhea through their effects on the immune system and resistance to colonization by pathogens. Also, there is some experimental evidence that certain Bifidobacteria may actually protect the host from the carcinogenic activity of intestinal flora. However, Bifidobacteria and Lactobacillus are the major bacteria that produce lactic acid and overabundance of these genera can cause a lactic acidosis. To have health benefits and prevent lactic acidosis, maintaining a certain number of Bifidobacteria and Lactobacillus genera is crucial.

As shown in FIGS. 12A and 12C, all six samples showed a significant reduction of Bifidobacteria. For Lactobacillus, except cucumber seed oil, five samples showed a significant increase or reduction. Therefore, these samples may be used to treat or prevent lactic acidosis.

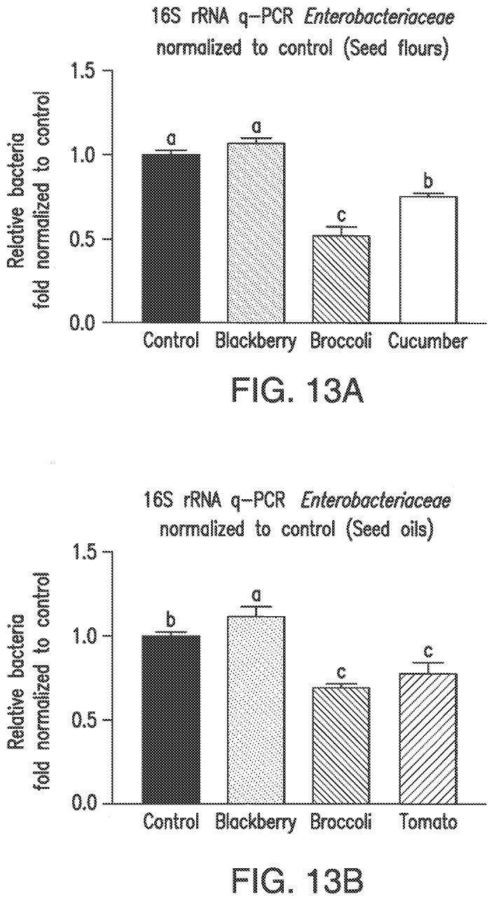

FIGS. 13A-13B depict the changes in the GUT microbiota profile for Enterobacteriaceae genus, for the tomato seed flour samples (FIG. 13A) and tomato seed oil samples (FIG. 13B).

Enterobacteriaceae genus is known as “bad” bacteria because they are pro-inflammatory pathobionts. Therefore, reducing the Enterobacteriaceae's population may have various health beneficial effects. In the present method, four samples including broccoli and cucumber seed flours and broccoli and tomato seed oils showed a significant reduction in the Enterobacteriaceae genus.

Summarizing the obtained results, it was validated that tomato seed oils and flours were able to alter the GUT microbiota profile in various ways. Therefore, blackberry, broccoli, and tomato seed oils and blackberry, broccoli, cucumber seed flours are prebiotic food products. As prebiotic foods, regular consumption of seed prebiotics may have several health beneficial effects on human health promotion and disease prevention.

The phenolic content and radical scavenging capacities of dietary ingredients are indicators of their usefulness as a source of antioxidants. In addition, recent studies suggest that phenolic compounds are able to interact with gut microbiota. Tomato seed flour, a byproduct of tomato seed oil production, was extracted using 50% acetone and tested for gut microbiota profile alteration, total phenolic content and radicals scavenging capacities. Tomato seed flour extract had 1.97 mg gallic acid equivalent/g (GAE/g) and RDSC, ORAC and ABTS□+ scavenging capacities of 3.81, 86.32 and 3.58 μmol Trolox equivalent (TE)/g, respectively. Also, tomato seed flour extract altered the GUT microbiota profile in vitro. The results suggest the potential for the use of tomato seed flour as value-added food ingredients. Identifying tomato seed flour as a value-added product can reduce waste and increase profits for businesses while improving human health.

The phenolic content and radical scavenging capacities of dietary ingredients are important for their usefulness as a source of antioxidants. Tomato seed flour and oil were tested for total phenolic content and radical scavenging capacities to identify their potential value-added food utilization. Two tomato seed flours and two tomato seed oils were tested for their total phenolic content and free radical scavenging capacities against DPPH⋅ and ABTS⋅+ radicals.

The results showed that tomato seed flour and tomato seed oil may contain significantly different amounts of phenolic compounds and free radical scavenging agents. In all three tests, tomato seed flour showed greater amounts of beneficial compounds than the tomato seed oil. The tomato seed oil samples were not significantly different from each other, while the two tomato seed flour samples were significantly different by total phenolic content; both were still significantly different than the tomato seed oil. The results suggest the potential for the use of tomato seed oil and tomato seed flour as value-added food ingredients. Identifying value-added components and properties product in tomato seeds can reduce waste and increase profits for tomato production and processing businesses while improving human health.

The subject method also addresses the study of the chemical composition of the tomato seed flour extract. The experimental studies have been conducted with two separate batches of tomato seed flour extracts TSF1 and TSF2, which have been analyzed for their chemical compositions, total phenolic content, and potential health benefits, particularly free radical scavenging capacities, anti-inflammatory capacities, and gut microbiota profile modulation which add further details to the evaluation of the total phenolic content, and potential health benefits, particularly free radical scavenging capacities, anti-inflammatory capacities, and gut microbiota profile modulation ability of the tomato seed flour presented in previous paragraphs.

The findings could serve as a scientific basis for the development of food products using tomato seed flours to improve human health, as well as further investigation of the biological benefits and molecular mechanisms behind it.

Chemical Composition of the Tomato Seed Flour Extract

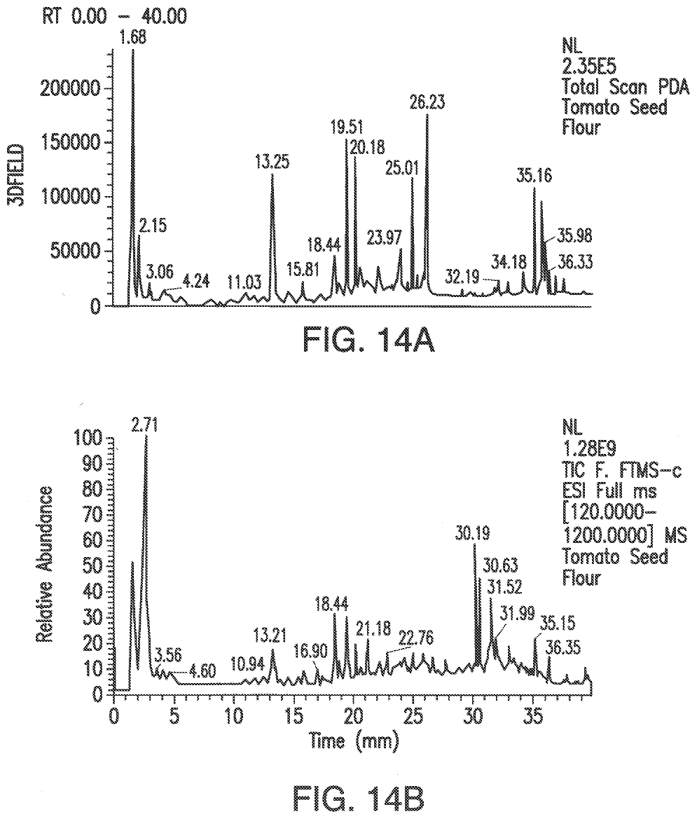

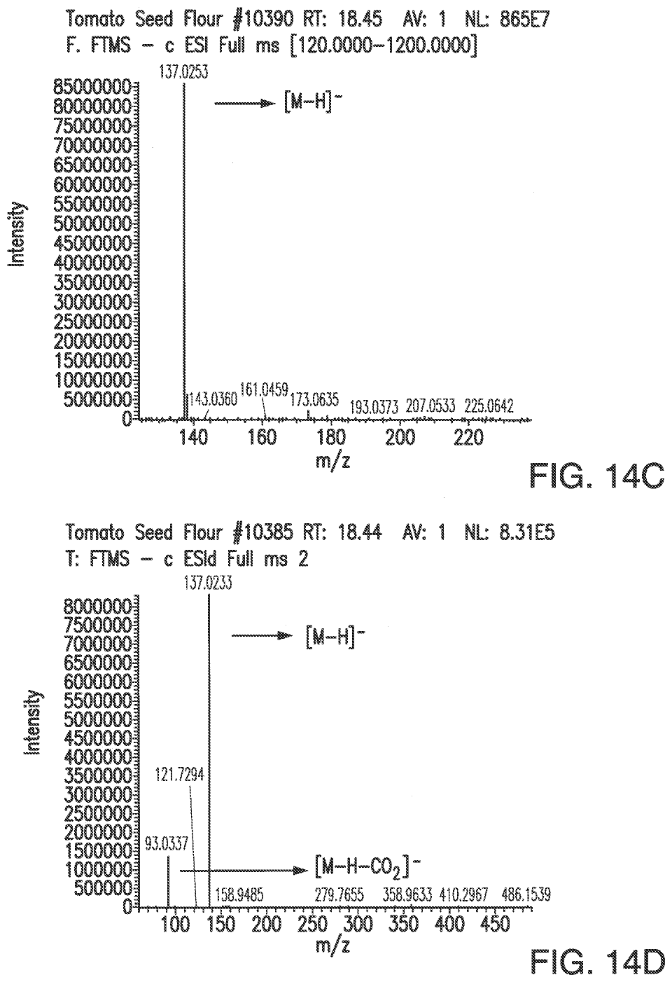

In both tomato seed flour extracts, named TSF1 and TSF2, a total of eight compounds, namely malic acid, 2-hydroxyadipic acid, salicylic acid, naringin, N-acetyl-tryptophan, quercetin-di-O-hexoside, kaempferol-di-O-hexoside, and azelaic acid were tentatively identified as shown in Table 2 and FIGS. 14A-14D.

TABLE 2

| | | | | | | Mass | |
|---|---|---|---|---|---|---|---|
| | Rt | Exptl. | | | | error | Tentative |
| ID | (min) | [M—H]⁻ | Fragment ions | | Formula | (mmu) | identification |
| 1 | 1.68 | 133.0135 | 71.0131 | | C₄H₆O5 | −0.75 | Malic acid |
| 2 | 4.95 | 161.0448 | 323.097 | ([2M—H]⁻) | C₆H₁₀O₅ | −0.75 | 2-Hydroxyadipic acid |
| | | | 143.0343 | ([M—H₂O]⁻) | | | |
| 3 | 18.44 | 137.0237 | 93.0337 | ([M—H—CO₂]⁻) | C₇H₆O₃ | −0.72 | Salicylic acid |
| 4 | 18.81 | 579.1708 | 271.0595 | ([narigenin—H]⁻) | C₂₇H₃₂O₁₄ | −1.13 | Naringin |
| 5 | 19.46 | 245.0919 | 491.1914 | ([2M—H]⁻) | C₁₃H₁₄N₂O₃ | −1.27 | N-Acetyl-tryptophan |
| 6 | 19.48 | 625.1381 | 300.0259 | ([quercetin-H]^{-•}) | C₂₇H₃₀O₁₇ | −2.92 | Quercetin-di-O-hexoside |
| | | | 301.0337 | ([quercetin-H]⁻) | | | |
| 7 | 20.14 | 609.1426 | 284.0310 | ([kaempferol-H]^{-•}) | C₂₇H₃₀O₁₆ | −3.51 | Kaempferol-di-O- |
| | | | 285.0389 | ([kaempferol-H]⁻) | | | hexoside |
| 8 | 21.17 | 187.0991 | 375.1929 | ([2M—H]) | C₉H₁₆O₄ | 1.52 | Azelaic acid |

Rt, retention time; Exptl. [M—H]⁻, experimental m/z of molecular ion.

FIG. 14A depicts a typical UHPLC-PDA chromatogram, while FIG. 14B depicts the total ion current (TIC) chromatogram of tomato seed flour extract, FIGS. 14C and 14D are representative of MS and MS/MS spectra, respectively, of peak 3 at the retention time of 18.44 min.

The tentative identification of the eight peaks were based on the theoretical, experimental molecular ions ([M-H]−) and the major MS/MS fragment ions, along with the MS data in the published literatures. For example, peak 3 had a m/z [M-H]− of 137.0233, which refers to the formula, C7H6O3 (mass error, −0.72 mmu) (FIG. 14C). The MS/MS spectrum showed m/z of 137.0233, which is was a parent ion and the fragmental ion had a peak of m/z 93.0337, resulting from a loss of the elimination of a carboxyl group from the molecular ion (FIG. 14D). This fragmentation matched with that previously reported for salicylic acid, indicating that peak 3 could be tentatively identified as salicylic acid.

Similarly, peak 1 of the tomato seed flour extract showed a m/z [M-H]− of 133.0135, which corresponds to the formula of C4H6O5 (mass error, −0.75 mmu) (Table 2). The peak 1 had the fragment ion at m/z of 71.0131 (Table 2). These m/z values were tentatively identified as malic acid. Same approach was applied to identify the other compounds found in tomato seed flour extract. As a result, 2-hydroxyadipic acid, naringin, N-acetyl-tryptophan, quercetin-di-O-hexoside, kaempferol-di-O-hexoside, and azelaic acid were tentatively identified.

Among eight identified compounds, four compounds including salicylic acid, naringin, quercetin-di-O-hexoside and kaempferol-di-O-hexoside are polyphenolic compounds. Polyphenolic compounds are known to possess potential health beneficial properties such as reducing the risk of arthritis, cancers, diabetes, obesity, and cardiovascular diseases.

Salicylic acid is a phenolic acid commonly found in many plants. It is widely used in medicines treating for skin redness. In addition, salicylic acid is also a metabolite of aspirin (acetylsalicylic acid), a pharmaceutical compound.

Naringin, quercetin-di-O-hexoside, and kaempferol-di-O-hexoside are polyphenol glycosides. In many cases, polyphenols found in plants exist as glycosides, in which a sugar is bound to another functional group through a glycosidic bond. In polyphenol glycosides, the sugar moiety is often hydrolyzed by the digestive enzymes or gut microflora and separated into sugar (glycone) and polyphenol (aglycone). Therefore, it is important to understand and evaluate both intact and hydrolyzed forms of polyphenols in terms of potential health beneficial properties. For example, naringin is a flavonoid and polyphenol glycoside found in fruits, vegetables and herbs, and will be hydrolyzed to naringenin, the aglycone of naringin in vivo.

Both naringenin and naringin have antioxidant capacities but compared to naringin, naringenin showed higher antioxidant capacity. In addition, naringenin possesses lipoprotein-lowering capacity. Similarly, both quercetin-di-O-hexoside and kaempferol-di-O-hexoside will be hydrolyzed to independent aglycones during digestion.

Quercetin is a flavonoid and has a number of health beneficial properties including free radical scavenging capacities, anti-tumor activities, and prevention of cardiovascular and neurodegenerative diseases.

A kaempferol is also a flavonoid found in plant food sources such as kale, spinach, and broccoli, and is known for its anti-cancer activity. The anti-cancer activity of kaempferol is mainly by modulating cell signals related to programmed cell death, new blood vessel formation, and spread of cancer cells.

The in vivo health beneficial properties of hydrolyzed polyphenols are important. But it is also important to note that the glycoside forms of polyphenolic compounds may contribute potential health beneficial properties such as free radical scavenging capacities, anti-inflammatory capacities, and gut microbiota modulation. For instance, glycoside forms of kaempferols including kaempferol-7-0-glucoside, kaempferol-3-O-rhamnoside, and kaempferol-3-O-rutinoside show anti-proliferative capacities, free radical scavenging capacities, and anti-inflammatory capacities. Also, glycoside forms of kaempferol, quercetin, and naringin are subjects to hydrolyzed by gut microbiota and may contribute to gut health.

In addition, four non-phenolic compounds were found in the tomato seed flour extract. These compounds are malic acid, 2-hydroxyadipic acid, N-acetyl-tryptophan, and azelaic acid as presented in Table 2.

Malic acid is an organic acid widely found in fruits and vegetables and contributes in the sour taste. Besides, malic acid can work as a bioavailability enhancer of minerals such as iron.

Tryptophan is an essential amino acid and involved in human health conditions including kidney diseases, cardiovascular diseases, diabetes, depression, sleep, and social behavior. In the current method, tryptophan was identified as an acetyl form. N-acetyl-tryptophan can also act as a tryptophan throughout the metabolism.

Azelaic acid is a carboxylic acid widely found in grains such as rye, oat, barley, wheat, and sorghum. Azelaic acid possesses protective effects against oxidative stress in several organs.

Thus, tomato seed flours have the potential for its utilization as food additives or functional food ingredient since tomato seed flours contain various health beneficial compounds.

Total Phenolic Content and Free Radical Scavenging Capacities of Tomato Seed Flour Extracts

Total phenolic content (TPC) is often closely connected to the free radical scavenging capacity and other potential health beneficial properties. In the current method, two different batches of tomato seed flour extracts, TSF1 and TSF2, showed TPC values of 2.00 and 1.97 mg gallic acid equivalents/g of seed flour (mg GAE/g), respectively, as presented in Table 3.

TABLE 3

| | | ORAC | DPPH | ABTS |
|---|---|---|---|---|
| | TPC mg | μmoles | μmoles | μmoles |
| Sample | GAE/g DW | TE/g DW | TE/g | TE/g DW |
| TSF1 | 2.00 ± 0.11 | 88.57 ± 2.42 | 3.57 ± 0.09 | 3.39 ± 0.08 |
| TSF2 | 1.97 ± 0.30 | 86.32 ± 7.01 | 3.81 ± 0.20 | 3.58 ± 0.61 |

In Table 3, TSF1 is the tomato seed flour extract from the first batch, TSF2 is the tomato seed flour extract from the second batch, TPC is the total phenolic content, ORAC stands for oxygen radical absorbing capacity, DPPH stands for 2,2-diphenyl-1-picrylhydrazyl radical scavenging capacity, ABTS stands for 2,2′-azino-bis(3-ethylbenzothiazoline-6-sulfonic acid) cation radical scavenging capacity, GAE stands for gallic acid equivalents, DW stands for dry weight, TE stands for Trolox equivalents.

Studies of the TPC of tomato seeds using three different tomato cultivars (including Excell, Tradiro and Flavourine) have been performed by others, and resulted in TPC values of 0.35, 0.29, and 0.21 mg GAE/g fresh weight, respectively. Compared to those TPC values, TSF1 and TSF2 in the subject study showed greater TPC values. This may due to different samples and extraction methods.

In the present method, 50% acetone was used to extract the tomato seed flour, while the earlier study used whole tomato seeds with 100% hexane and a combination of acetone, water, and acetic acid (70:29.5:0.5, v/v/v) as solvents for lipophilic and hydrophilic compounds, respectively. The TSF1 and TSF2 in the subject method also resulted in greater TPC values than the TPC of a tomato fruit (about 0.68 mg GAE/g) of earlier studies.

For free radical scavenging capacities, TSF1 and TSF2 showed oxygen radical absorbing capacities (ORAC), 2,2-diphenyl-1-picrylhydrazyl radical scavenging capacities (DPPH), and 2,2′-azino-bis(3-ethylbenzothiazoline-6-sulfonic acid) cation radical scavenging capacities (ABTS) of 88.57-86.32, 3.57-3.81, 3.39-3.58 μmoles Trolox equivalents/g of seed flour (μmoles TE/g), respectively (Table 3).

Compared to DPPH or ABTS, ORAC values were much greater. These differences may due to the different structure of the radicals and reactivities between radical and polyphenolic compounds found in tomato seed flour extracts. During the reaction of free radical scavenging by antioxidants, radicals are quenched by two mechanisms in most cases, namely hydrogen atom transfer (HAT) or single electron transfer (SET). The mechanisms of ABTS and DPPH assays are based on mixed HAT and SET, however, the ORAC's mechanism is solely based on HAT. So, the antioxidant capacity maybe different based on different assays, therefore, two or more assays are needed in evaluating a selected antioxidant compound.

Earlier studies by others evaluated the free radical scavenging capacities of ten tomatoes grown in Colorado and found ABTS values in the range of 5.4-20.9 and μmoles TE/g dry weight. They also reported TPC values of ten tomatoes. TPC values were in the range of 2.9-5.0 mg GAE/g. Compared to the current study's TPC and ABTS values, both values were greater for tomatoes grown in Colorado. These results suggest possible correlation between the TPC and free radical scavenging capacities, which was also confirmed by the Pearson correlation analyses in the present study.

As a result, TPC showed significantly positive correlation with ORAC (r=0.975, P≤0.01), DPPH (r=0.991, P≤0.01), and ABTS (r=0.987, P≤0.01) values, suggesting that total phenolics in tomato seed may directly contribute to the radical scavenging capacities.

In earlier studies by others, free radical scavenging capacities of two tomato varieties, Grape and Saladette, were evaluated. It was found that ORAC and DPPH values of 1631.39-2426.53, 307.8-465.17 μmoles TE/g dry weight, respectively. Overall, tomato seed flours had lower free radical scavenging capacities per dry weight basis compared to tomato fruits.

Anti-Inflammatory Capacities of Tomato Seed Flour Extracts

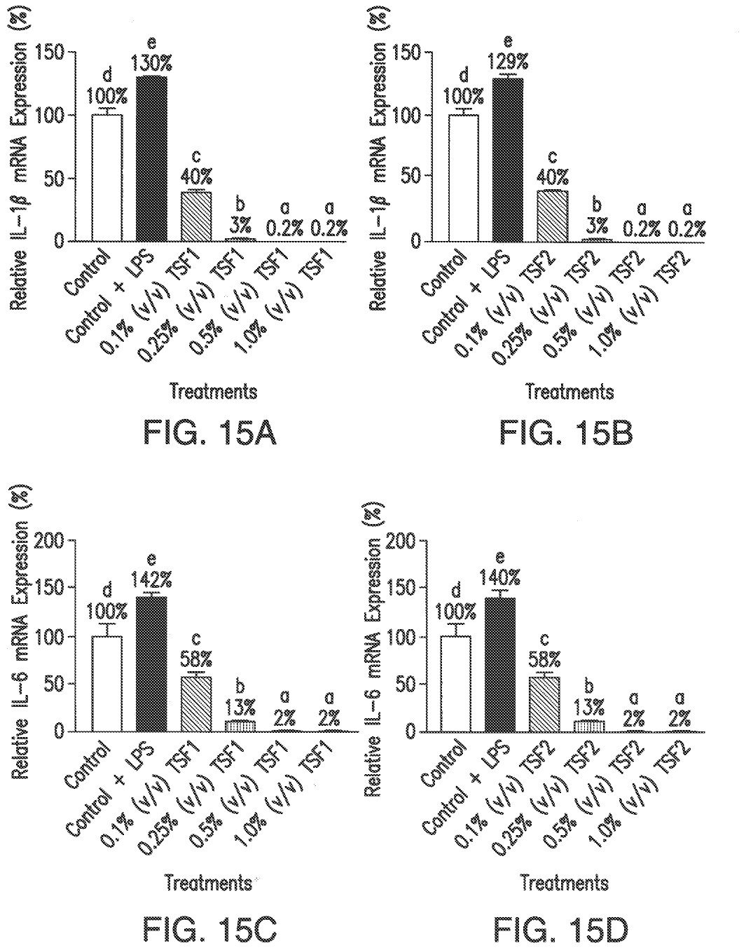

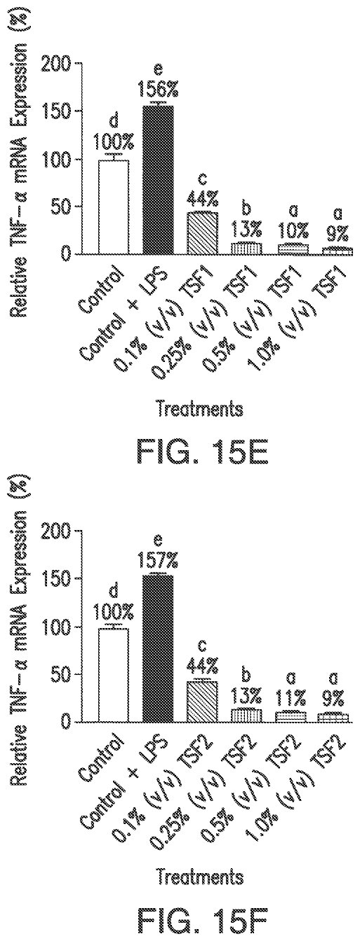

In the present study, TSF1 and TSF2 both demonstrated dose-dependent anti-inflammatory capacities against pro-inflammatory markers including interleukin-1 beta (IL-1β), interleukin-6 (IL-6) and tumor necrosis factor-alpha (TNF-α) presented in FIGS. 15A-15F, where FIGS. 15A, 15C, 15E are representative of the first batch of tomato seed flour extract, and FIGS. 15B, 15D, 15F are representative of the second batch of tomato seed flour extract.

The treatments of different concentrations from 0.1% to 1.0% v/v of TSF1 and TSF2 were capable of inhibiting the lipopolysaccharide (LPS) stimulated IL-1p mRNA expression by 69, 97, 99, 99, 69, 97, 99 and 99%, respectively. Similarly, TSF1 and TSF2 dose dependently inhibited IL-6 and TNF-α mRNA expressions under the experimental conditions. It needs to be pointed out that all the tomato seed flour extracts with different concentrations used in this test had no adverse effects on THP-1 macrophages viability according to the MTT assay.

The anti-inflammatory capacities of tomato using macrophages differentiate from THP-1 monocytes and LPS as a stimulant. It is known that the tomato extract is able to inhibit gene expressions of pro-inflammatory markers such as IL-1p and TNF-α. However, the anti-inflammatory capacities of tomato seed flour have not been evaluated in the past. Hence, the current method evaluated it for the first time.

The inflammation process is often closely related to free radicals and chronic diseases. Free radicals are produced by a variety of sources within the human body. Sources are often divided into two groups, endogenous and exogenous sources. Endogenous sources are mitochondria, peroxisomes and phagocytes, while exogenous sources are cigarette smoking, air pollution, radiation, some medications and ozone. The primary cause of chronic diseases is oxidative stress, an imbalance between antioxidants and free radicals.

In the human body, there is a transcription factor named nuclear factor erythroid-derived 2-like 2 (NRF2) that modulates the production of antioxidants and detoxifying products. These products can protect the damage caused by oxidative stress. But this may not be enough to protect the body from oxidative stress, and the antioxidants from food sources are important for overall antioxidative status in the human body.

When oxidative stress is induced, immune cells start to react. Among many immune cells, macrophages perform a central role in inflammation and start to secret pro-inflammatory cytokines and tumor necrosis factors such as IL-1β, IL-6, and TNF-α. Bioactive compounds in foods can reduce or inhibit the development of these pro-inflammatory cytokines and tumor necrosis factor. For example, kaempferol can act as a nuclear factor kappa-light-chain-enhancer of activated B cells (NF-KB) inhibitor by binding its pathway signaling protein.

Quercetin inhibites cytokine and inducible nitric oxide synthase expressions through inhibiting the NF-κB pathway. Moreover, naringenin, aglycone of naringin, has been reported to inhibit enzymes responsible for pro-inflammatory responses such as cyclooxygenase-1 (COX-1) and cyclooxygenase-2 (COX-2).

The results obtained in the present study regarding the anti-inflammatory capacities of polyphenolic compounds suggest that tomato seed flour has potential anti-inflammatory capacities and may be utilized for food additives or functional foods for improving human health.

Gut Microbiota Profile Modulation of Tomato Seed Flour Extracts

Maintaining a healthy gut microbiota profile is a key to maintaining a good state of health. Each organism, however, has a different gut microbiota profile, and in addition, along with the aging process, the gut microbiota profile changes. For example, a naturally delivered baby and a cesarean delivered baby may have different gut microbiota profiles.

Also, each individual has a different metabolism rate. Therefore, the speed of the aging process may be distinct. These intrinsic factors are not changeable.

On the other hand, there are extrinsic factors that can shift the gut microbiota profile. These factors include exercise, stress, antibiotics, and diet. Among these extrinsic factors, the diet seems to have the biggest impact.

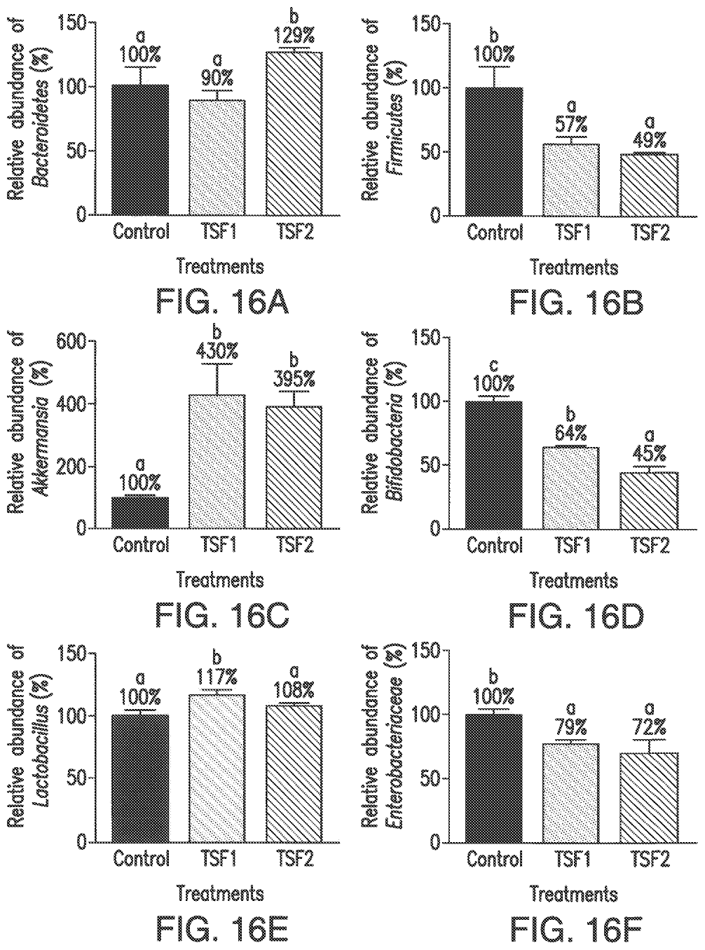

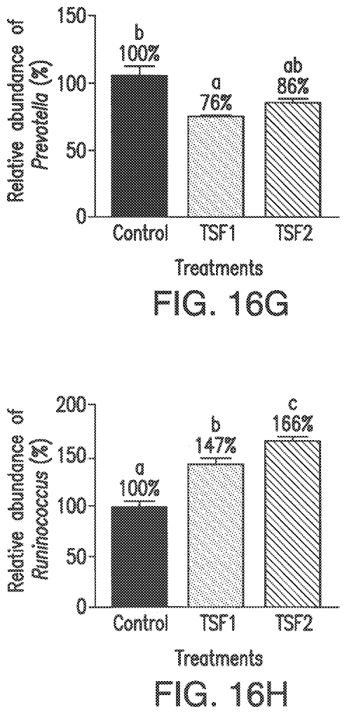

FIGS. 16A-16H depict the GUT microbiota profile modulation by tomato seed flour extracts with regard to Bacteroidetes phylum (FIG. 16A), Firmicutes phylum (FIG. 16B), Akkermansia genus (FIG. 16C), Bifidobacterium genus (FIG. 16D), Lactobacillus genus (FIG. 16E), Enterobacteriaceae genus (FIG. 16F), Prevotella genus (FIG. 16G), and Ruminococcus genus (FIG. 16H).

In the current method, a total of eight bacterial taxonomic ranks including Bacteroidetes phylum, Firmicutes phylum, Akkermansia genus, Bifidobacterium genus, Lactobacillus genus, Enterobacteriaceae genus, Prevotella genus, and Ruminococcus genus were used to evaluate the gut microbiota profile change by tomato seed flour extracts. Five phyla and genera showed significant changes (P≤0.05). Among five significantly changed phylum or genus, the abundance of Akkermansia and Ruminococcus genera were increased (FIGS. 16C and 16H) and Firmicutes phylum, Bifidobacterium genus, and Enterobacteriaceae genus were decreased (FIGS. 16B, 16D and 16H). Even though one batch (TSF2) significantly increased the Bacteroidetes phylum, the other batch (TSF1) did not show any significant effect (FIG. 16A). Also, the Lactobacillus genus was significantly increased by one batch (TSF1), but not by TSF2 (FIG. 16E).

Cumulatively, these results suggest a possible variation between tomato seed samples in their microbiota modulating properties. In vivo study is needed to further confirm the effects of tomato seed flour on Bacteroidetes phylum and Lactobacillus genus.

GUT microbiota profile modulation by other vegetable seed flours including broccoli, carrot, and cucumber has been investigated. In that study, the abundance of Bacteroidetes phylum was significantly increased by cucumber seed flour extract and decreased by carrot seed flour extract and not changed by broccoli seed flour extract. In the current study, one batch of tomato seed flour extract (TSF2) significantly increased Bacteroidetes phylum but the fold was much lower than that of cucumber seed flour extract.

Furthermore, both batches of tomato seed flour extracts decreased the abundance of Firmicutes phylum (FIG. 16B). This Firmicutes phylum decrease has been also observed in a study, where broccoli, carrot, and cucumber seed flour extracts significantly decreased the abundance of Firmicutes. Also, a similar trend was observed in the Enterobacteriaceae genus which was significantly decreased (FIG. 16F).

For probiotic bacteria, Bifidobacterium and Lactobacillus genera, only the Bifidobacterium genus was decreased by TSF1 and TSF2 (FIGS. 16D and 16E). This was different from the observations in the previous study where all three vegetable seed flour extracts decreased both probiotic bacteria, Bifidobacterium and Lactobacillus genera.

In GUT microbiota, Bacteroidetes and Firmicutes phyla consist of more than 90% population and play important roles. Bacteroidetes can activate lymphocyte T cell to modulate human immune responses and produce butyric acid. In addition, Bacteroidetes are participating in the conversion of toxin and carcinogen and bile acid metabolism. Firmicutes are closely related to the aging process and are possibly involved in fatty acid metabolism. Bifidobacterium and Lactobacillus genera are probiotics and known to reduce infectious diarrhea and pathogen colonization. Akkermasia are a mucin degrading bacterium and maintaining gut health, and could reduce body fat mass and adipose tissue inflammation and improve glucose homeostasis. Prevotella can be used as a biomarker for gut dysbiosis since the abundance of the Prevotella genus is associated with diets rich in plants and their components such as carbohydrates and fibers. Ruminococcus possess abilities to breakdown and use a wide range of plant polysaccharides for host health. Since the tomato seed flour extracts significantly increased both Akkermansia and Ruminococcus genera, tomato seed flour may be used as a functional food for weight control and improving digestion.

Chemicals and Reagents

Tomato seed flours were donated by the Botanic Innovations (Spooner, Wis., USA). PMA (phorbol 12-myristate 13-acetate) (product#: P1585), Trolox (6-Hydroxy-2,5,7,8-tetramethylchroman-2-carboxylic acid) (product#: 238813), gallic acid (product#: G7384), Folin-ciocalteu reagent (2N) (product#: F9252), and sodium carbonate (product#: 223530) were acquired from Sigma Aldrich (Saint-Louis, Mo., USA). AAPH (2,2′-Azinobis (2-amidinopropane) dihydrochloride) (catalog#: 992-11062) was acquired from Wako Chemicals (Richmond, Va., USA).

Materials for the cell culture were purchased from GIBCO (Grand Island, N.Y., USA). Materials for PCR (polymerase chain reaction) were acquired from either Thermo Fisher Scientific (Fair Lawn, N.J., USA) or Qiagen (Gaithersburg, Md., USA).

Tomato Seed Flour Extract Preparation

For extraction, 10 g of tomato seed flour was mixed with 50 mL of 50% acetone and vortexed for 1 min. Then, the mixture was sonicated for 1 min and rested for 24 hours. This extract was used to assess free radical scavenging capacities, anti-inflammatory capacities, and GUT microbiota profile modulation.

In addition, alternative solvents, including ethanol/water and acetone/water at ratios ranging from 100:0 to 0:100 (v/v), may be used to prepare the seed derivatives extracts using reflux, perculation, soaking and/or Soxhlet extraction methods, with the subsequent removal of the solvent(s) and water.

For the chemical composition analysis, 10 g of tomato seed flour was extracted using Soxhlet extractor with 50 mL of 100% ethanol.

Ultra-High-Performance Liquid Chromatography-High Resolution Mass Spectrometry (UHPLC-HRMS) Analysis

The UHPLC-HRMS (Themo Scientific, Waltham, Mass.) system consists of an Orbitrap ID-X tribrid mass spectrometer with a Vanquish UHPLC including a high-pressure binary pump, thermostatting column temperature control compartment, and an HL Diode Array Detector [58]. The separation was carried out on an Agilent RRHD Eclipseplus C18 2.1*150 mm 1.8 μm (Agilent, Palo Alto, Calif.) with an UltraShield pre-column filter (Analytical Scientific Instruments, Richmond, Calif.) with a flow rate of 0.3 mL·min-1. Solution A (0.1% formic acid in water, v/v) and solution B (0.1% formic acid in acetonitrile, v/v) was used for gradient elution with the following program.

The proportion remained at 2% B (v/v) at 0-5 min, and subsequently increased to 10% B at 15 min, to 45% B at 25 min, to 90% at 35 min, and this proportion remained at 90% up to 40 min.

The post-run time for re-equilibration was 10 min. The UV-vis spectra were recorded at the range of 190-600 nm for the entire run. The column temperature was set at 50° C. and the sample compartment temperature was set at 4° C. The injection volume was 2 μL.

The MS conditions were set as follows: sheath gas at 50 (arbitrary units), auxiliary gas at 10 (arbitrary units), and sweep gas at 1 (arbitrary units), spray voltage at 3 kV with negative ionization mode, ion transfer tube temperature at 300° C., vaporizer temperature at 350° C., RF lens at 60%.

The full scan mass ranged from 120 to 1200 m/z with a resolution of 60,000, AGC target value of 200,000 in full scan and 10, 000 FTMS/MS, and max ion injection time of 50 ms. The most intense ion was selected for the data-dependent scan with normalization collision energy at 35% in HCD. Data were post-processed using the Xcalibur 2.2 software.

Total Phenolic Content

Total phenolic content was evaluated by the laboratory procedure as follows:

3 mL of deionized water, 50 μL of sample, standard, or solvent (blank), and 250 μL of the diluted Folin-Ciocalteu reagent (0.5 N) were added to the test tube and vortexed for 1 min. After vortexing, 750 μL of 20% (w/v) sodium carbonate was added to trigger the reaction.

After 2 hours, wavelength of 765 nm was used to measure the absorbance. The result was expressed in milligrams of gallic acid equivalents (GAE) per gram of the seed flour sample.

Relative 2,2-Diphenyl-1-Picrylhydrazyl (DPPH) Radical Scavenging Capacity

The relative DPPH radical scavenging capacity was examined using the wavelength of 515 nm for measuring the absorbance every minute. The absorbance was recorded for 40 min. To provide a standard curve, the Trolox was used. For the value unit, micromoles of Trolox equivalents/g of flour (μmol TE/g) was used.

Oxygen Radical Absorbing Capacity (ORAC)

The oxygen radical absorbing capacity (ORAC) was also measured in the present method. In order to generate a standard curve, different concentrations of the Trolox was dissolved in 50% acetone. All other reagents were prepared using 75 mM pH 7.4 phosphate buffer. For the detection, wavelengths of 485 and 535 nm were used for the excitation and emission, respectively. The ORAC value was reported as μmol TE/g of the flour samples.

2,2′-Azinobis (3-Ethylbenzothiazoline-6-Sulphonic Acid) Diammonium Salt Cation Radical (ABTS⋅+) Scavenging Capacity

The ABTS cation radical scavenging capacity was evaluated using the following protocol:

ABTS⋅+ working solution was prepared by oxidizing ABTS with manganese oxide and absorbance was adjusted to 0.700±0.005 at 734 nm. Trolox was used as the antioxidant standard. For the reaction, 1 mL of ABTS⋅+ working solution was mixed with 80 μL of the sample, standard, or solvent. This mixture was vortexed for 30 s. After 90 s, the absorbance value was recorded at 734 nm. The ABTS value was reported as μmol TE/g of the flour samples.

Anti-Inflammatory Capacity

To assess the anti-inflammatory capacity, THP-1 macrophages were used. The density of 6×105 cells/mL THP-1 macrophages were cultured in six-well plates to achieve 80% confluence. Subsequently, macrophages were cultured with and without the tomato seed flour extracts at concentrations of 0.1, 0.25, 0.5, and 1.0% v/v for 24 hours.

For the stimulation, 10 ng/mL of lipopolysaccharide (LPS) was used. After 4 hours of stimulation with LPS, cells were lysed for RNA isolation. From RNA, cDNA was synthesized and Real-time PCR was performed using TaqMan probe. TATA binding protein (TBP) was used as a control primer, and IL-1β, IL-6, and TNF-α were used as inflammatory markers.

Gut Microbiota Analysis

A regular chow diet-fed C57BL/6J mouse's fecal sample was used to prepare gut microbiota. For the bacterial concentration calculation, OD600 value of 1=8×10⁸ cells/mL was used.

1×10⁷ cells/mL of bacterial cells were cultured in M9 broth with and without tomato seed flour extract in 15 mL and 50 mL tubes for 6 hours with shaking.

After 6 hours, bacterial cells were collected by centrifugation at 5000 rpm for 5 min. Bacterial DNA were extracted using Precellys lysing and QIAamp DNA mini kits.

Specific forward and reverse primer sequences used in this study were shown as follows:

Akkermansia
(Forward: 5-CAGCACGTGAAGGTGGGGAC-3′,
Reverse: 5′-CCTTGCGGTTGGCTTCAGAT-3′);
Bacteroidetes
(Forward: 5′-GGARCATGTGGTTTAATTCGATGAT-
3′, Reverse: 5′-AGCTGACGACAACCATGCAG-3′);
Bifidobacterium
(Forward: 5′-TCGCGTCYGGTGTGAAAG-3′,
Reverse: 5′-CCACATCCAGCRTCCAC-3′);
Enterobacteriaceae
(Forward: 5′-CATTGACGTTACCCGCAGAAGAAGC-3′,
Reverse: 5′-CTCTACGAGACTCAAGCTTGC-3′);
Firmicutes
(Forward: 5′-GGAGYATGTGGTTTAATTCGAAGCA-3′,
Reverse: 5′- AGCTGACGACAACCATGCAC-3′);
Lactobacillus
(Forward: 5′-GAGGCAGCAGTAGGGAATCTTC-3′,
Reverse: 5′-GGCCAGTTACTACCTCTATCCTTCTTC-3′);
Prevotella
(Forward: 5′-TCCTAGGGAGGCAGCAGT-3′,
Reverse: 5′-CAATCGGAGTTCTTCGTG-3′);
and
Ruminococcus
(Forward: 5′-GGCGGCCTACTGGGCTTT-3′,
Reverse: 5′-CCAGGTGGATAACTTATTGTGTTAA-3′).

Real-time PCR was carried with the SYBR probe.

The current study observed numerous potential health beneficial polyphenolic compounds in tomato seed flour extracts, along with several possible health beneficial properties including free radical scavenging capacities, anti-inflammatory capacities, and gut microbiota profile modulation. The results might be used to improve human health by increasing the utilization of tomato seed flour as a food additive or functional food ingredient.

Although this invention has been described in connection with specific forms and embodiments thereof, it will be appreciated that various modifications other than those discussed above may be resorted to without departing from the spirit or scope of the invention as defined in the appended claims. For example, functionally equivalent elements may be substituted for those specifically shown and described, certain features may be used independently of other features, and in certain cases, particular locations of the elements may be reversed or interposed, all without departing from the spirit or scope of the invention as defined in the appended claims.

What is claimed is:

1. A method for modulating microbiota in the gastrointestinal tract (GUT) of a user, comprising:
processing seeds of at least one plant selected from a group including fruits, vegetables, berries, and a combination thereof, thus producing at least one seed derivative selected from a group including a seed powder/meal, a seed oil, a seed flour, a seed powder/meal extract, a seed flour extract, a seed oil extract, and a combination thereof, wherein said at least one seed derivative is characterized by a Total Phenolic Content, and free radical scavenging and anti-inflammation capacities;
validating biological effects of said at least one seed derivative for the ability to modulate the GUT microbiota; and
consuming said at least one seed derivative by the user to result in modulating the GUT microbiota profile, said modulation of the GUT microbiota profile resulting in a reduction of oxidative stress, inflammation, and risk of chronic diseases through the interaction of the phenolic content and free radical scavenging and anti-inflammatory capacities of said at least one seed derivative with the GUT microbiota.

2. The method of claim 1, wherein said at least one plant includes a tomato, and said at least one seed derivative includes at least one of tomato seed powder/meal, tomato seed flour, tomato seed oil, and a combination thereof.

3. The method of claim 2, further comprising:
preparing a tomato seed flour sample extract for said validation of biological effects of the tomato seed flour on the GUT microbiota, the tomato seed flour sample extract preparation including:
weighting a predetermined amount of a tomato seed flour sample, and extracting said tomato seed flour sample extract a plurality of consecutive times, each extraction with at least one solvent selected from a group including 25-50 mL of 50% acetone, a solution of ethanol/water and acetone/water at ratios ranging from 100:0 to 0:100 (v/v), and a combination thereof, by a an extracting routine selected from a group including reflux, percolation, soaking, Soxhlet extraction routines, and a combination thereof.

4. The method of claim 3, further comprising:
preparing the GUT microbiota complex containing Bacteroidetes and Firmicutes phyla, and Akkermansia, Bifidobacteria, Enterobacteriaceae, Lactobacillus, Prevotella and Ruminococcus genera of said GUT microbiota complex reacted with the tomato seed flourextract,
validating the biological effects of the tomato seed flour on the GUT microbiota by applying the 16S rRNA gene sequencing to Bacteroidetes and Firmicutes phyla, and Akkermansia, Bifidobacteria, Enterobacteriaceae, Lactobacillus, Prevotella and Ruminococcus genera in said GUT microbiota complex reacted with said tomato seed flour sample extract.

5. The method of claim 4, wherein said 16S rRNA gene sequencing further includes the steps of:
treating said GUT microbiota complex with 0.1% of the tomato seed flour sample extract,
extracting bacterial DNA from said GUT microbiota complex treated with the tomato seed flour sample extract,
performing Real-Time Polymerase Chain Reaction (PCR) with a reaction system containing 10 μL SYBR®Green Real-SCR Master Mix, 0.25 μL 500 nM oligo primers, 4.5 μL water, and 5 μL of said bacterial DNA, and
determining a relative content of said Bacteroidetes and Firmicutes phyla, and Akkermansia, Bifidobacteria, Enterobacteriaceae, Lactobacillus, Prevotella and Ruminococcus genera in said reaction system.

6. The method of claim 3, further comprising:
validating the biological effects by measuring Total Phenolic Content (TPC) of the tomato seed flour sample extract by:
analyzing the TPC of a reaction mixture of the tomato seed flower sample extract and gallic acid by the Folin-Ciocalten colorimetric method,
measuring the absorbance of said reaction mixture of the tomato seed flour sample extract and gallic acid at 765 nm, and
expressing the TPC as mg gallic acid equivalent (GAE) per gram of the tomato seed flow sample extract.

7. The method of claim 3, further comprising:
determining the chemical composition of the tomato seed flour extract by:
obtaining a typical UHPLC-PDA chromatogram and the total ion current (TIC) chromatogram of the tomato seed flour extract, and
identifying 8 peaks from said chromatograms, said 8 peaks correlating with malic acid, 2-hydroxyadipic acid, salicylic acid, naringin, N-acetyl-tryptophan, quercetin-di-O-hexoside, kaempferol-di-O-hexoside, and azelaic acid.

8. The method of claim 3, further comprising:
validating the biological effects including the free radical scavenging and anti-inflammatory capacities of the tomato seed flour sample extract.

9. The method of claim 8, further comprising:
validating the free radical scavenging capacity by a method selected from a group comprising: oxygen absorbing capacity (ORAC), relative 2,2-diphenyl-1-picrylhydrazyl (DPPH) radical scavenging capacity (RDSC), ABTS⋅+ scavenging capacity, and a combination thereof.

10. The method of claim 8, further comprising:
validating the anti-inflammatory capacity by evaluation of inflammatory response of interleukin-beta (IL-1β), interleukin-6 (IL-6), and tumor necrosis factor alpha (TNF-d) inflammation markers reacted with the tomato feed flour sample extract.

11. The method of claim 2, further comprising:
preparing a tomato seed oil sample extract,
preparing the GUT microbiota complex containing Bacteroidetes and Firmicutes phyla, and Akkermansia, Bifidobacteria, Enterobacteriaceae, Lactobacillus, Prevotella and Ruminococcus genera, and
validating the biological effects of the tomato seed oil sample extract on the GUT microbiota by applying the 16S rRNA gene sequencing to Bacteroidetes and Firmicutes phyla, and Akkermansia, Bifidobacteria, Enterobacteriaceae, Lactobacillus, Prevotella and Ruminococcus genera in said GUT microbiota complex reacted with the tomato seed oil sample extract.

12. The method of claim 4, wherein the validation of the biological effects includes a detection of a significant increase of the ratio between Bacteroidetes and Firmicutes phyla, thus proving a potential of consumption of the tomato seed flour in controlling body weight gain and reducing the risk of obese-related chronic diseases.

13. The method of claim 4, wherein the validation of the biological effects includes detection of an increase of Bacteroidetes phylum, and decrease of Firmicutes phylum, thus proving a potential of consumption of the tomato seed flour in health-beneficial effects related to nutrition, xenobiotic, drug metabolism, antimicrobial protection, and immune enhancement.

14. The method of claim 4, wherein the validation of the biological effects includes detection in an increase in Alkermansi genus, thus proving a potential of consumption of the tomato seed flour in reduction of the risk of developing obesity and type 2 diabetes and the increasing possibility of reversing obesity and type 2 diabetes.

15. The method of claim 4, wherein the validation of the biological effects includes detection of:
a reduction of Bifidobacteria genus and increase of Lactobacillus genus, thus proving a potential of consumption of the tomato seed flow in preventing infectious diarrhea, carcinogenic activity, and treating lactic acidosis,
reduction in Enterobacteriaceae genus, thus proving a potential of consumption of tomato seed flour in reduction of pro-inflammatory pathobionts, and
reduction in Prevotella genus and increase in Ruminococcus genus, thus proving a potential of consumption of tomato seed flow in lowering the risk of chronic inflammatory disease.

16. The method of claim 10, wherein the validation of the anti-inflammatory capacity of the tomato seed flour includes detection of suppression of mRNA-expressions of the pro-inflammation genes including IL-1β, IL-6, and TNF-α, thus proving a potential of consumption of the tomato seed flour in treating inflammation and inflammation related chronic diseases.

17. The method of claim 9, wherein the validation of the free radicals scavenging capacities of the tomato seed flour sample extract against said ORAC, DPPH and ABTS assays result in the levels of 86.3-88.6, 3.6-3.8 and 3.4-3.6 μmoles (TE)/g, respectively, thus proving a potential of the consumption of the tomato seed flour in scavenging free radicals.

18. The method of claim 6, wherein said tomato seed flour extract has the TPC of 1.97-2.00 mg gallic acid equivalent/g (GAE/g).

19. A seed-based food additive for modulating microbiota in the gastrointestinal tract (GUT) of a user, comprising:
a seed derivative selected from a group including a seed oil, a seed flour, seed powder/meal, seed oil extract, seed flour extract, seed powder/meal extract, and a combination thereof, prepared by the processing of at least one plant selected from a group including fruits, vegetables, berries, and a combination thereof,
wherein said seed derivative includes malic acid, 2-hydroxyadipic acid, salicylic acid, naringin, N-acetyl-tryptophan, quercetin-di-O-hexoside, kaempferol-di-O-hexoside, and azelaic acid, and
wherein said seed derivative is characterized by:
(a) an increased Total Phenolic Content (TPC) ranging from 1.97 to 2.00 mg GAE/g beneficial in free radicals scavenging capacity of the seed derivative,
(b) an ability to increase a ratio between Bacteroidetes and Firmicutes phyla of the GUT microbiota beneficial in controlling body weight gain and reducing the risk of obese-related chronic diseases, and
(c) an ability to increase Bacteroidetes phylum and to decrease Firmicutes phylum, beneficial in promoting health-beneficial effects related to nutrition, xenobiotic, drug metabolism, antimicrobial protection, and immune enhancement.

20. The seed-based food additive of claim 19, wherein said food additive is further characterized by the ability to:
increase Akkermansia genus in the GUT microbiota to reduce the risk of obesity and type 2 diabetes,
to reduce Bifidobacteria genus and increase Lactobacillus genus to prevent infectious diarrhea and carcinogenic activity, and to treat lactic acidosis,
to reduce Enterobacteriaceae genus in the GUT microbiota to reduce pro-inflammatory pathobionts,
to reduce Prevotella genus and to increase Ruminococcus genus in the GUT microbiota to prevent a risk of chronic inflammatory disease, to suppress pro-inflammatory genes, and to treat inflammation related chronic diseases, and
wherein said seed-based food additive has free radicals scavenging capacity evaluated against ORAC, DPPH and ABTS assays of the levels of 86.3-88.6, 3.6-3.8 and 3.4-3.6 μmoles (TE)/g, respectively.
